(19) United States
(12) Patent Application Publication
(10) Pub. No.: US 2026/0200889 A1
(43) Pub. Date: Jul. 16, 2026

(54) CONTINUOUS-FLOW CATALYTIC DEHYDRATION PROCESS FOR THE SYNTHESIS OF MACROCYCLIC COMPOUNDS

(71) Applicant: Indian Institute of Science Education and Research, Pune (IISER Pune), Pune (IN)

(72) Inventors: Boopathy GNANAPRAKASAM, Pune (IN); Dashrat Vishambar SUTAR, Pune (IN); Akash Bandu JAMDADE, Pune (IN)

(73) Assignee: Indian Institute of Science Education and Research, Pune (IISER Pune), Pune (IN)

(21) Appl. No.: 19/364,834

(22) Filed: Oct. 21, 2025

(30) Foreign Application Priority Data
Oct. 21, 2024 (IN) 202421079997

(51) Int. Cl. C07D 323/00; C07C 41/01; C07D 273/01; C07D 327/10

(52) U.S. Cl. CPC C07D 323/00; C07C 41/01; C07D 273/01; C07D 327/10

(57) ABSTRACT

The present invention relates to a continuous-flow catalytic dehydration process for the synthesis of macrocyclic compounds. More specifically, the present invention relates to a process of synthesizing macrocyclic ether, macrolactone and macrolactam compounds of formula (I) in good yields and also demonstrated in gram scale application. Further, the process of the present invention of synthesizing new macrocyclic compounds generates only water as a by-product. The advantages of the process of present invention include heterogeneous catalysis and scalable to grams, diverse substrates with different ring sizes, water as a by-product, no requirement of predefined substrates, and no special reaction conditions.

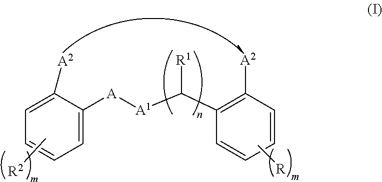

### FIELD OF THE INVENTION

The present invention relates to organic chemistry. Specifically, the present invention relates to a continuous-flow catalytic dehydration process for the synthesis of macrocyclic compounds. More specifically, the present invention relates to a process for synthesizes of macrocyclic ether, macrolactone and macrolactam derivatives in good yields and is also demonstrated in gram scale application. Further, the process of the present invention of synthesizing new macrocyclic compounds generates only water as a by-product.

### BACKGROUND OF THE INVENTION

Background description includes information that may be useful in understanding the present invention. It is not an admission that any of the information provided herein is prior art or relevant to the presently claimed invention, or that any publication specifically or implicitly referenced is prior art.

Macrocyclic compounds are significant synthetic targets due to their wide applications in catalysis, agrochemicals, cosmetics, and the pharmaceutical industry. This scaffold is found in many biologically active natural products and ~20% of natural products from earthbound and marine sources possess macrocyclic structures. Notably, macrocyclic compounds found in natural products show biological applications and are used in drug discovery, particularly to address the difficult protein-protein interactions. Thus, macrocycles are very important skeletons in the pharmaceutical industry. In this class, macrocyclic polyethers, macrolactones, and macrolactams have found distinct applications in different fields, especially polycyclic ethers show applications in ion selectivity and phase transfer catalysis. In addition, various macrocyclic ether or polyethers have special cavity diameters that offer better binding with cancer cells and serve as supramolecular receptors via the formation of host-guest complexes. Numerous macrolactones are used in the perfume industry and crown ethers are used in supramolecular chemistry. Moreover, macrolactams are ubiquitous structural cores which are found in many drugs and natural products. Regardless of their applications, synthesis of these macrocyclic compounds is typically very challenging due to the higher entropy and also non-selective due to the formation of a mixture of oligomers, which results in poor yields and less efficiency. Considering the accomplishment in inter or intramolecular macrocyclic etherification via nucleophilic substitution reactions (Williamson etherification), pre-activation of the alcohol substrates such as halides, mesylates, or tosylate derivatives were prepared (FIG. 1′a). This traditional approach requires a stoichiometric base, genotoxic halides/mesylate/tosylate as substrates, and copious waste formation, making them non-sustainable and less atom-economical reactions. On the other hand, very few catalytic macrocyclic etherifications have been reported in the last few decades. In 2001, Decicco et al reported Cu-mediated macrocyclic biphenyl ether synthesis using intramolecular O-arylation of phenols with phenylboronic acid pseudopeptides (FIG. 1′b). A series of macrocyclic ethers, including natural products, were synthesized by fewer research groups using the Cu-catalysed intramolecular Ullmann coupling reaction of phenol and aryl halides under harsh reaction conditions (FIG. 1′c). An intramolecular oxidative coupling of phenols has been reported for the synthesis of macrocyclic aryl ether using PbO₂ (FIG. 1′d). All these methods were accessed for the synthesis of macrocyclic aryl ether via intramolecular C—O bond formation. The indirect approaches for the synthesis of macrocyclic alkyl ether include intramolecular ring-closing alkene metathesis of specially designed alkenyl acyclic ether in the presence of Grubbs catalyst (FIG. 1′e). The aforementioned catalytic approaches necessitate specially designed substrates (alkene, boronic ester, aryl halides, phenols), multistep synthesis and an expensive non-recyclable metal catalyst is required, which leads to copious genotoxic stoichiometric waste. Therefore, an opportunity remains for efficient catalytic macrocyclization with high selectivity and sustainability to avoid the activators and specially designed substrates/conditions.

The dehydration of alcohols promoted by a catalyst is a synthetically useful reaction for the construction of C—C, C—O and C—N bonds with complementary advantages such as step-economy, atom-economy, absence of activator/special reactant/condition and generating water as a greener by-product. Furthermore, homogeneous catalytic systems have been well studied for the direct etherification from alcohols via dehydrative coupling. Many of these kinds of catalytic reactions are efficient in forming acyclic ether and small ring cyclic ether. However, there is no report on intramolecular catalytic dehydrative macrocyclization using symmetrical and unsymmetrical diol (FIG. 7).

Within the domain of macrocycles, crown ethers, macrolactones and macrolactams are very important macrocycles that exhibit different biological activities. In the literature, there is no report on the synthesis of macrocyclic ethers, macrolactones and macrolactams using reusable heterogeneous catalysts.

Macrocyclic polyether is commercially made by using diols and halides, which subsequently generate copious waste. There are catalytic reactions that need specially designed starting materials. However, there is no report on intramolecular catalytic dehydrative macrocyclization using symmetrical and unsymmetrical diol.

Macrocycles are very important skeletons in the pharmaceutical industry. Among several macrocyclic ethers, crown ethers are an important class of macrocycles that exhibit broader biological applications. Numerous macrolactones are used in the perfume industry and crown ethers are used in supramolecular chemistry. Moreover, macrolactams are ubiquitous structural cores which is found in many drugs and natural products. Despite their applications, the synthesis of these macrocyclic compounds is an entropically forbidden process and also non-selective due to the formation of a mixture of oligomers, which results in poor yields and less efficiency. However, there are several approaches for the synthesis of macrocyclic ether, macrolactone and macrolactam derivatives using homogeneous catalysts under batch conditions. Typically, the macrocyclization reactions work well at higher dilution conditions and the main challenge associated with homogeneous macrocyclization is the large-scale synthesis due to the difficulty in handling high dilution. Also, it is difficult to recover and reuse homogeneous catalysts.

Therefore, there is an unmet need in the art to develop a new sustainable process to synthesize macrocyclic compounds that overcome one or more drawbacks of the prior arts.

### OBJECTIVE OF THE INVENTION

An objective of the present invention is to provide a continuous-flow catalytic dehydration process for the synthesis of macrocyclic compounds of formula (I).

Another objective of the present invention is to provide a process that synthesizes several macrocyclic ether derivatives, macrolactones and macrolactams compound of formula (I) in good yields and to demonstrate the process for the gram scale application.

Another objective of the present invention is to provide a process of synthesizing new macrocyclic compounds of formula (I) that generate only water as a by-product.

### SUMMARY OF THE INVENTION

The present invention relates to a continuous-flow catalytic dehydration process for the synthesis of macrocyclic compounds. More specifically, the process synthesizes several macrocyclic ether derivatives, macrolactones and macrolactams in good yields and is also demonstrated in gram scale application. Further, the process of the present invention of synthesizing new macrocyclic compounds generates only water as a by-product.

In an aspect, the present invention relates to a continuous flow process for preparation of macrocyclic compound of formula (I),

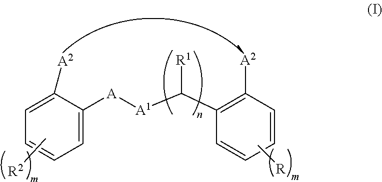

wherein:
A is CH₂ or CO;
A¹ is O or NH;
A² is O or S;
R is hydrogen, OH, C₁₋₆alkyl, halogen, substituted or unsubstituted phenyl, or C₁₋₆alkyloxy; or R along with hydrogen of phenyl ring form naphthyl ring, wherein the substitution is one or more group selected from halogen or OH;
R¹ is hydrogen, or C₁₋₆alkyl;
R² is hydrogen, OH, C₁₋₆alkyl, halogen, substituted or unsubstituted phenyl, or C₁₋₆alkyloxy, or R² along with hydrogen of phenyl ring form naphthyl ring, wherein the substitution is one or more group selected from halogen or OH;
n is 0, 1, 2, 3, 4 or 5;
m is 0, 1 or 2; and
[symbol] represents C₂₋₁₄ linear alkyl; wherein the one or more CH₂ of C₂₋₁₄ linear alkyl is optionally replaced with O or S.
wherein the process comprises the steps of:
dissolving a compound of formula (Ia) (wherein X is CH₂OH, COOH or CONH₂;
A², R, R¹, R², n and m are as defined above) in a solvent to obtain a solution; and

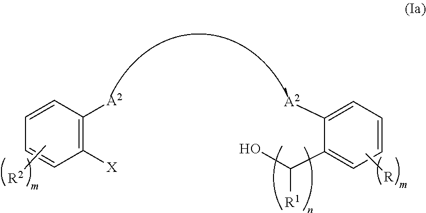

reacting the solution with a heterogeneous catalyst present in a continuous flow reactor at a temperature in the range of 100° C. to 150° C. with the flow rate in a range of 0.05 mL/min to 2 mL/min at back pressure in a range of 2.5 to 4 bar to obtain the compound of formula (I).

In another aspect of the present invention, A² is O.

In another aspect of the present invention, A² is S.

In another aspect, the present invention provides a process for the intramolecular dehydration of diols, seco-acids, and amido-alcohols for the synthesis of macrocyclic compounds in the presence of a heterogeneous catalyst under the continuous-flow module as given in the scheme below.

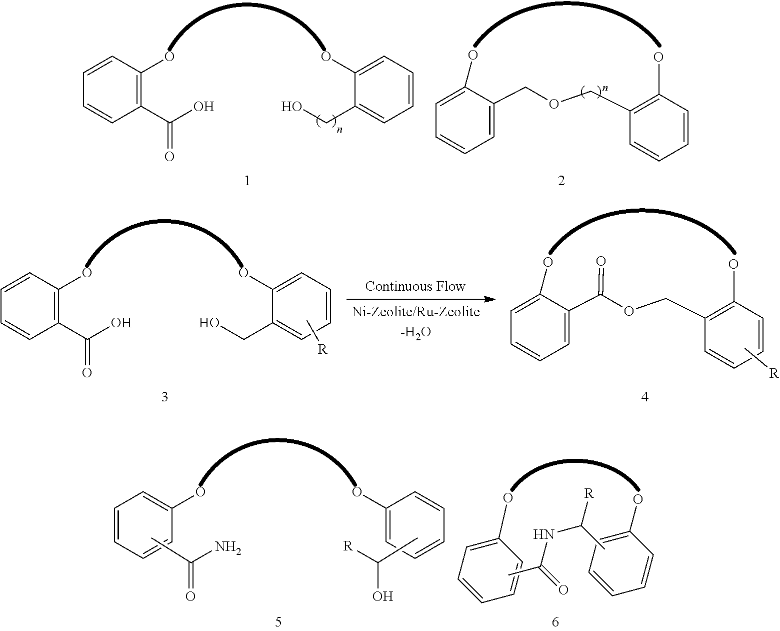

In another aspect of the present invention, macrocyclic compounds are selected from macrocyclic polyether, macrocyclic lactones, and macrocyclic lactams.

In another aspect of the present invention, the solvent is selected from the group consisting of toluene, 1,4-dioxane, acetonitrile, tetrahydrofuran, 2-methyltetrahydrofuran, dimethyl sulfoxide, dimethylformamide, dimethyl carnonate, and diethylene glycol dimethyl ether.

In another aspect of the present invention, the catalyst is selected from β-Zeolite, Amberlyst-15, Amberlyst-35, Nafion, montmorillonite clay, dowex 50, Ni-Zeolite, Fe-Zeolite, Ru-Zeolite, NiCl₂·6H₂O, RuCl₃·xH₂O, SnCl₂, ZnCl₂, FeCl₃, Fe(OTf)₃, TiCl₄, InCl₃, and Mn(OAc)₃.

In another aspect of the present invention, the diols are selected from symmetrical diols and unsymmetrical diols.

In another aspect, the present invention relates to Ni-Zeolite catalyzed dehydrative coupling of diols to form novel macrocyclic polyethers under continuous flow process.

In another aspect, the present invention relates to continuous flow intramolecular dehydrative coupling to form the macrocyclic lactone from the seco-acid using Ru-Zeolite.

In another aspect, the present invention relates to continuous flow intramolecular dehydrative coupling to form the macrocyclic lactams from the amido-alcohols using Ru-Zeolite.

In another aspect of the present invention, Ni-Zeolite and Ru-Zeolite are recyclable catalysts.

In another aspect, the process of the present invention for delivering a new class of macrocycles (ether, ester and amide) can be used in host-guest chemistry and drug discovery.

Various objects, features, aspects and advantages of the inventive subject matter will become more apparent from the following detailed description of preferred embodiments.

### BRIEF DESCRIPTION OF FIGURES

FIG. 1 illustrates the customized continuous flow setup. (1) Temperature, (2) Product, (3) Omnifit column bed, (4) BPR, (5) Starting material, (6) Heating block, and (7) Peristaltic pump.

FIG. 2 illustrates the catalyst and solvent studies in batch and continuous flow for the macrolactonization.

FIG. 3 illustrates the temperature and dilution studies in batch and continuous flow for the macrolactonization.

FIG. 4 illustrates the Ni-Zeolite catalyst reusability under continuous-flow

FIG. 5 illustrates the Ni-Zeolite catalyst time run study under continuous-flow.

FIG. 6 illustrates the recyclability of Ru-Zeolite catalyst for macrolactamization under continuous-flow.

FIG. 7 illustrates the state-of-the-art for macrocyclic etherification.

FIG. 8 illustrates scheme 1 of the macrocyclic etherification as per the exemplary embodiments of the present invention.

FIG. 9 illustrates scheme 2 of the macrocyclic etherification as per the exemplary embodiments of the present invention.

FIG. 10 illustrates scheme 3 of the macrocyclic etherification as per the exemplary embodiments of the present invention.

FIG. 11 illustrates scheme 4 of the macrocyclic etherification as per the exemplary embodiments of the present invention.

FIG. 12 illustrates scheme 5 of the macrocyclic etherification as per the exemplary embodiments of the present invention.

FIG. 13 illustrates scheme 6 of the macrocyclic etherification as per the exemplary embodiments of the present invention.

FIG. 14 illustrates scheme 7 of the macrocyclic etherification as per the exemplary embodiments of the present invention.

### DETAILED DESCRIPTION OF THE INVENTION

The following is a full description of the disclosure's embodiments. The embodiments are described in such a way that the disclosure is clearly communicated. The level of detail provided, on the other hand, is not meant to limit the expected variations of embodiments; rather, it is designed to include all modifications, equivalents, and alternatives that come within the spirit and scope of the current disclosure as defined by the attached claims. Unless the context indicates otherwise, the term “comprise” and variants such as “comprises” and “comprising” throughout the specification are to be read in an open, inclusive meaning, that is, as “including, but not limited to.”

When “one embodiment” or “an embodiment” is used in this specification, it signifies that a particular feature, structure, or characteristic described in conjunction with the embodiment is present in at least one embodiment. As a result, the expressions “in one embodiment” and “in an embodiment” that appear throughout this specification do not necessarily refer to the same embodiment. Furthermore, in one or more embodiments, the specific features, structures, or qualities may be combined in any way that is appropriate.

Unless the content clearly demands otherwise, the singular terms “a,” “an,” and “the” include plural referents in this specification and the appended claims. Unless the content explicitly mandates differently, the term “or” is normally used in its broad definition, which includes “and/or.”

In some embodiments, the numbers expressing quantities of ingredients, properties such as concentration, reaction conditions, and so forth, used to describe and claim certain embodiments of the invention are to be understood as being modified in some instances by the term “about.”

The recitation of ranges of values herein is merely intended to serve as a shorthand method of referring individually to each separate value falling within the range. Unless otherwise indicated herein, each individual value is incorporated into the specification as if it were individually recited herein.

All processes described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

The headings and abstract of the invention provided herein are for convenience only and do not interpret the scope or meaning of the embodiments.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description that follows, and the embodiments described herein, is provided by way of illustration of an example, or examples, of particular embodiments of the principles and aspects of the present disclosure. These examples are provided for the purposes of explanation, and not of limitation, of those principles and of the disclosure.

It should also be appreciated that the present invention can be implemented in numerous ways, including as a system, a method or a device. In this specification, these implementations, or any other form that the invention may take, may be referred to as processes. In general, the order of the steps of the disclosed processes may be altered within the scope of the invention.

Various terms as used herein are shown below. To the extent a term used in a claim is not defined below, it should be given the broadest definition persons in the pertinent art have given that term as reflected in printed publications and issued patents at the time of filing.

The term “C₂₋₁₄ linear alkyl” as used herein refers to the radical of saturated aliphatic groups, including straight chain alkyl groups having fourteen or fewer carbon atoms in its backbone, for instance, C₁-C₁₈ or C₁₋₆ for straight chain. As used herein, (C₂₋₁₄) alkyl refers to an alkyl group having from 1 to 14 carbon atoms. Representative examples of alkyl include, but are not limited to, methyl, ethyl, n-propyl, n-butyl, n-pentyl, n-hexyl, isopropyl, sec-butyl, isobutyl, tert-butyl, isopentyl, 2-methylbutyl and 3-methylbutyl.

The term, “halogen” as used herein refers to chlorine, fluorine, bromine or iodine atom.

The term, “(C₁₋₆)alkoxy” refers to a (C₁₋₆)alkyl having an oxygen radical attached thereto. Representative examples of alkoxy groups include, but are not limited to, methoxy, ethoxy, propoxy, isopropoxy, n-butoxy, isobutoxy and tert-butoxy. Furthermore, unless stated otherwise, the alkoxy groups can be unsubstituted or substituted with one or more groups. A substituted alkoxy refers to a (C₁₋₆)alkoxy substituted with one or more groups, particularly one to four groups independently selected from the groups indicated above as the substituents for the alkyl group.

In a general embodiment, the present invention relates to a continuous-flow catalytic dehydration process for the synthesis of macrocyclic compounds. More specifically, the process synthesizes several macrocyclic ether derivatives and macrolactone in good yields and is also demonstrated in gram-scale application. Further, the process of the present invention of synthesizing new macrocyclic compounds generates only water as a by-product.

In an embodiment, the present invention relates to a continuous flow process for preparation of macrocyclic compound of formula (I) from the compound of formula (Ia) in presence of heterogeneous catalyst as shown the general scheme below,

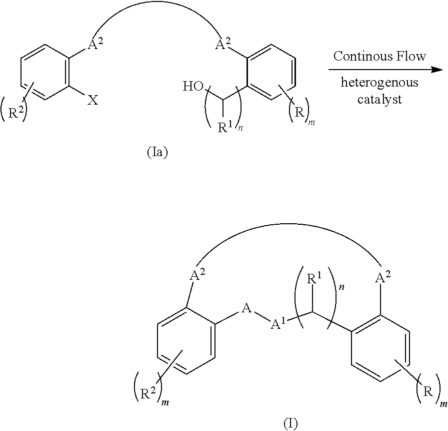

wherein:
X is CH₂OH, COOH or CONH₂;
A is CH₂ and CO;
A¹ is O and NH;
A² is O or S;
R is hydrogen, OH, C₁₋₆alkyl, halogen, substituted or unsubstituted phenyl, or C₁₋₆alkyloxy; or R along with hydrogen of phenyl ring forms naphthyl ring, wherein the substitution is one or more groups selected from halogen or OH;
R¹ is hydrogen, or C₁₋₆alkyl;
R² is hydrogen, OH, C₁₋₆alkyl, halogen, substituted or unsubstituted phenyl, or C₁₋₆alkyloxy, or R² along with hydrogen of phenyl ring form naphthyl ring, wherein the substitution is one or more groups selected from halogen or OH;
n is 0, 1, 2, 3, 4 or 5;
m is 0, 1 or 2; and
[symbol] represents C₂₋₁₄ linear alkyl; wherein one or more CH₂ of C₂₋₁₄ linear alkyl is optionally replaced with O or S.

In an embodiment, the present invention provides a process for the intramolecular dehydration of diols, seco-acids and amido-alcohols for the synthesis of macrocyclic compounds in the presence of a heterogeneous catalyst under continuous-flow module.

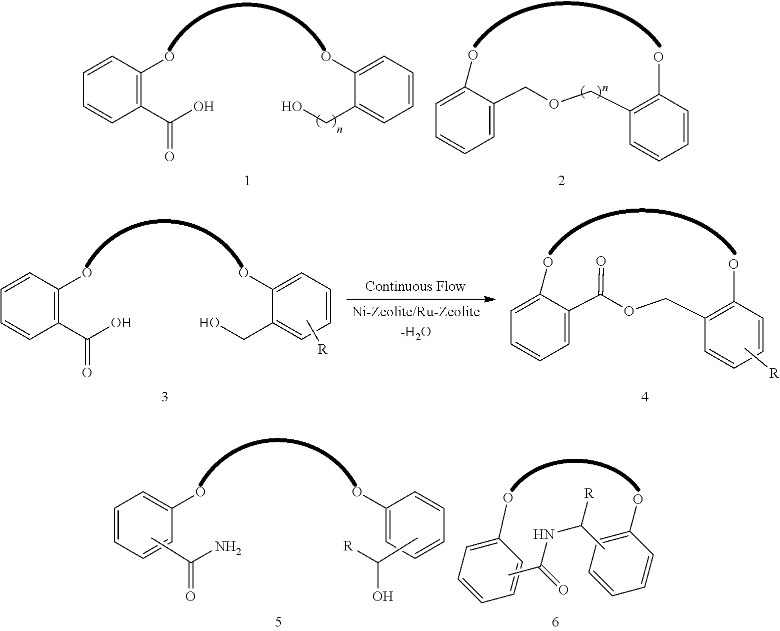

In another embodiment of the present invention, macrocyclic compounds are selected from macrocyclic polyether, macrocyclic lactones and macrocyclic lactams.

In another aspect of the present invention, the solvent is selected from the group consisting of toluene, 1,4-dioxane, acetonitrile, tetrahydrofuran, 2-methyltetrahydrofuran, dimethyl sulfoxide, dimethylformamide, dimethyl carnonate, diethylene glycol dimethyl ether Preferably, the solvent is dioxane and acetonitrile.

In another embodiment of the present invention, the catalyst is selected from β-Zeolite, Amberlyst-15, Amberlyst-35, Nafion, montmorillonite clay, Dowex 50. Ni-Zeolite, Fe-Zeolite, Ru-Zeolite, NiCl₂·6H₂O, RuCl₃·xH₂O, SnCl₂, ZnCl₂, FeCl₃, Fe(OTf)₃, TiCl₄, InCl₃, Mn(OAc)₃. Preferably, the catalyst is selected from Ni-Zeolite and Ru-Zeolite.

In another embodiment of the present invention, the continuous flow process is carried out at the temperature in the range of 115° C. to 125° C.

In another embodiment of the present invention, the flow rate of the continuous flow process is 0.1 mL/min.

In another embodiment of the present invention, the back pressure of the continuous flow process is in a range of 3.3 to 3.5 bar.

In another embodiment of the present invention, when X of the compound of formula (Ia) represents CH₂OH then A is CH₂ and A¹ is O in the compound of formula (I).

In another embodiment of the present invention, when X of the compound of formula (Ia) represents COOH then A is CO and A¹ is O in the compound of formula (I).

In another embodiment of the present invention, when X represents CONH₂ in the compound of formula (Ia) then A is CO and A¹ is NH in the compound of formula (I).

In another embodiment of the present invention, the [symbol] represents C₂₋₁₄ linear alkyl; wherein one or more CH₂ of C₂₋₁₄ linear alkyl is optionally replaced with O.

In another embodiment of the present invention, the diols are selected from symmetrical diols and unsymmetrical diols.

In another embodiment, the present invention relates to Ni-Zeolite catalyzed dehydrative coupling of diols to form novel macrocyclic polyethers under continuous flow. The ability of Ni-Zeolite as an efficient catalyst for the double dehydrative coupling to form C═C bond towards the macrocyclic alkenylation. Thus, the present invention provides an efficient intramolecular dehydrative coupling of symmetrical/unsymmetrical diols for the synthesis of macrocyclic crown ethers in the presence of Ni-Zeolite as a catalyst under continuous-flow catalysis.

In another embodiment, the present invention relates to continuous flow intramolecular dehydrative coupling of seco-acids to form the macrocyclic lactone using Ru-Zeolite. Thus, the method is efficient for the intramolecular dehydrative macrolactonization of seco-acids using Ru-Zeolite.

In another embodiment, the present invention relates to continuous flow intramolecular dehydrative coupling of amido-alcohols to form the macrocyclic lactam using Ru-Zeolite. Thus, the method is efficient for the intramolecular dehydrative macrolactamization of amido-alcohols using Ru-Zeolite.

In another embodiment of the present invention, the Ni-Zeolite and Ru-Zeolite are recyclable catalysts.

In another embodiment, the present invention provides a new method for macrocyclic etherification using heterogeneous Ni-Zeolite as a reusable catalyst under continuous flow chemistry.

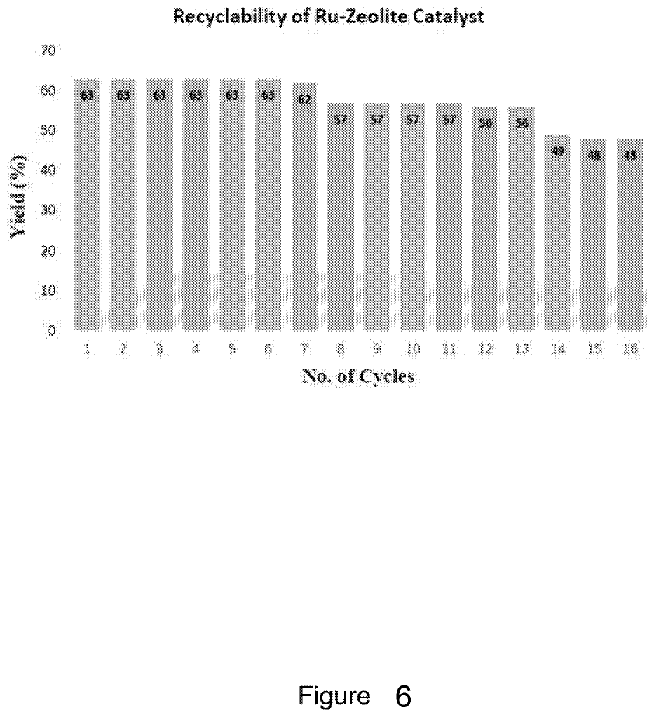

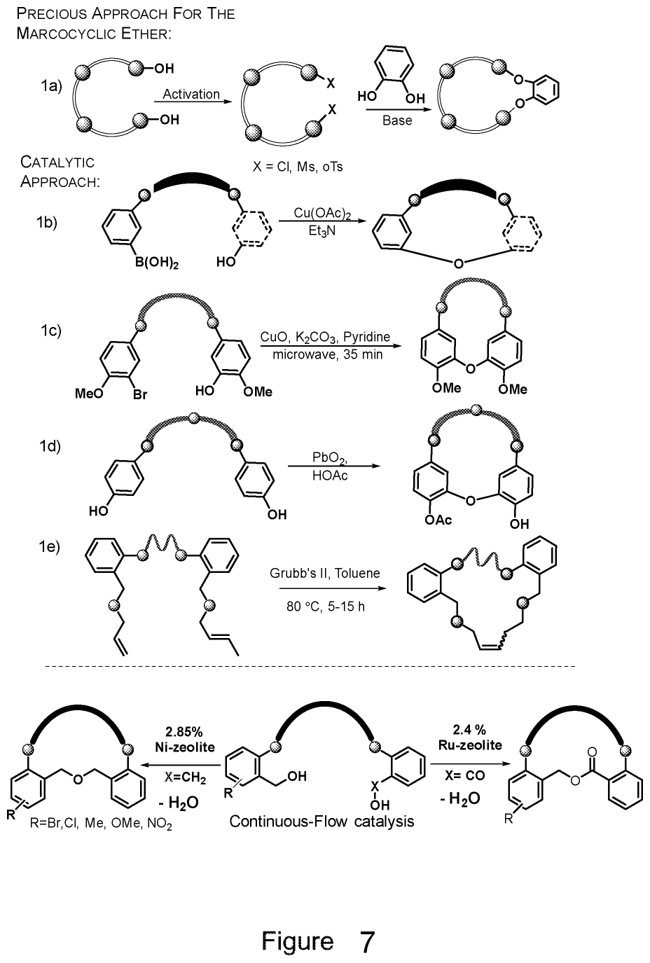

In yet another embodiment, the present invention provides continuous-flow catalysis for the intramolecular dehydration of diols, seco-acids and amido-alcohols for the synthesis of macrocyclic polyethers, lactones and lactams. For the macrocyclization, reusable heterogeneous Ni-Zeolite one-time pack cartridges are used, which effectively catalyses the intramolecular reaction of diols and generate a diverse array of new macrocyclic polyether with different ring sizes. In the case of macrocyclic lactone and macrocyclic lactam, Ru-Zeolite catalysed the seco-acid and amido-alcohol to produce a series of macrocyclic lactones and lactams with different ring sizes. This reaction was generalized to several substrates, demonstrated in the gram-scale and recyclability of the catalyst and solvent (FIG. 6).

In another embodiment of the present invention, the flow catalysis is generalized with wide substrates by one-time packed Ni-Zeolite to produce 20 macrocyclic polyethers, 11 examples of broad macrolactones and 15 examples of macrolactams with water as a byproduct. Several of these derivatives were prepared in good yields and also demonstrated in gram-scale application.

In another embodiment, the process of the present invention for delivering a new class of macrocycles (ether, ester and amide) can be used in host-guest chemistry and drug discovery.

While the foregoing describes various embodiments of the disclosure, other and further embodiments of the disclosure may be devised without departing from the basic scope thereof. The scope of the invention is determined by the claims that follow. The invention is not limited to the described embodiments, versions or examples, which are included to enable a person having ordinary skill in the art to make and use the invention when combined with information and knowledge available to the person having ordinary skill in the art.

### EXAMPLES

The present invention is further explained in the form of the following examples. However, it is to be understood that the following examples are merely illustrative and are not to be taken as limitations upon the scope of the invention.

### Example 1: Optimization for Macrocyclic Polyether

Macrocyclic etherification of the compound diphenyldimethanol 1a as a model substrate to provide the macrocyclic ether 2a by screening different solvents, temperatures, and catalysts in batch reaction conditions was done. Compound 1a (0.15 mmol) in solvents (2 mL, Table 1) and ^{a}50, ^{b}100, ^{c}150 mg of catalyst were stirred in a sealed cap vial at various temperatures for 24 h. The mentioned yields are isolated yields.

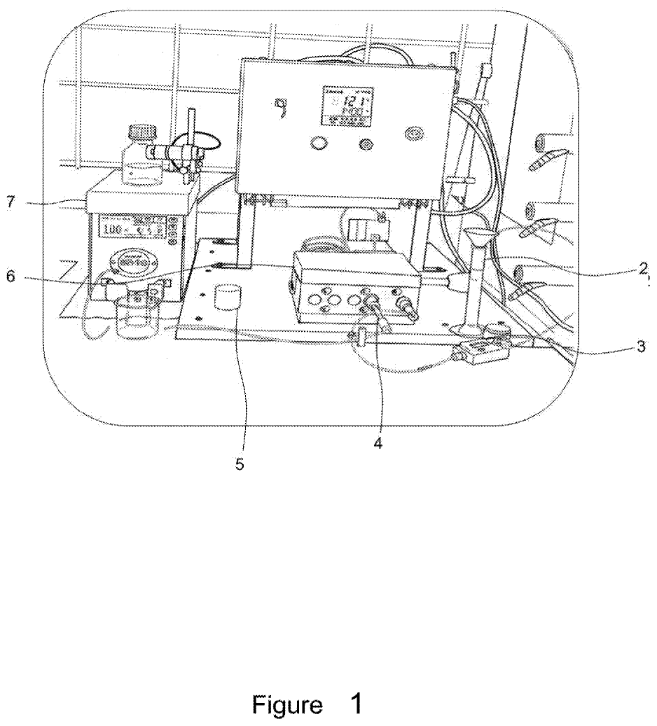

The reaction was optimized under fixed-bed continuous flow chemistry catalysis to address concerns about yield and scale-up problems associated with a batch process. For the continuous flow setup, a Vapourtec SF-10 peristaltic pump was used for pumping starting material in a solvent, an omnifit glass column bed for packing heterogeneous catalysts, a modified heating block for heating, perfluoro alkoxy alkane (PFA) tubing for reaction, and an adjustable back pressure regulator (BPR) from Vapourtec to maintain the pressure (FIG. 1) In the glass column (Omnifit® (6.6×150 mm) bed reactor was added 1.7 g of the catalyst and tightly closed by adjustable PFA adaptor. Then, this fixed bed column was connected to a heating jacket with the inlet and outlet tubing.

### Example 2: General Process for Intramolecular Macrocyclic Etherification and Gram Scale Synthesis of Macrocyclic Polyether

In 10-20 mL reagents vials were added 0.05 M solution of diol 1 in dioxane solvent was pumped using a syringe pump through a glass column (Omnifit® (6.6×150 mm) packed bed containing 1.7 g of Ni-zeolite filled up to 7 cm and preheated at 120° C. with the flow rate of 0.1 mL/min at 3.3 to 3.5 bar back pressure was maintained in the reaction and residence time t_{R}=27.3 min. After a four-time run. The collected organic layer was concentrated under reduced pressure and the crude product was purified by column chromatography using ethyl acetate:hexane (05:95 to 20:80) as an eluent to afford the compound 2 (FIG. 8, Scheme 1).

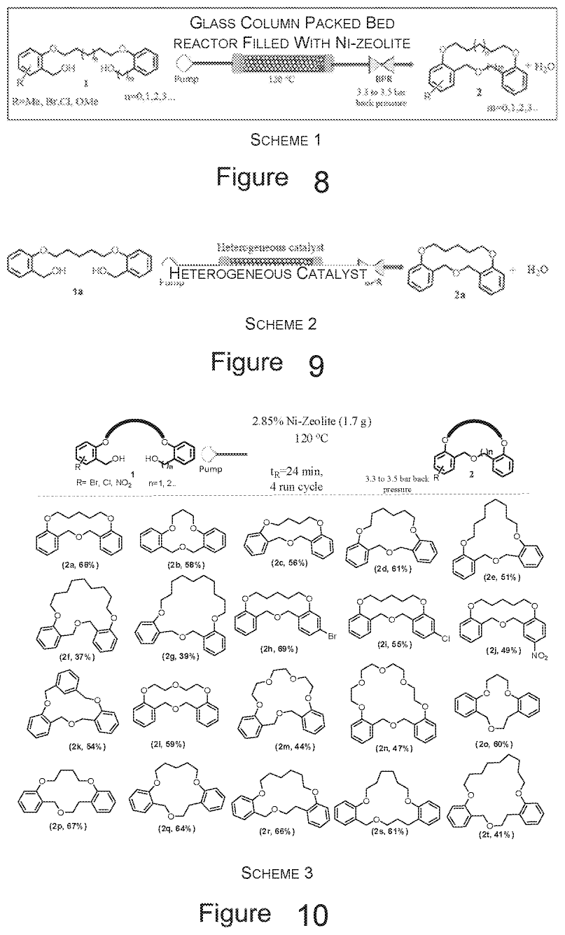

A solution of 1a in different solvents was prepared and flowed (0.1 mL/min flow rate) through the Omnifit column containing the heterogeneous catalysts (1.5 g, 5 cm height), at specified temperatures and residence time, (Table 1). ^{a}1, 4-dioxane, ^{b}THF, ^{c}benzene, ^{d}toluene was used as a solvent. ^{e}1.7 g, 7 cm height of catalysts used at 3.3 to 3.5 bar back pressure. The mentioned yields are isolated yields. In addition, this optimized reaction condition was also performed on a large scale (6.2 mmol of 1a) to furnish a 65% yield of compound 2a (FIG. 9, Scheme 2).

TABLE 1
Optimization for the macrocyclic ether in a continuous flow

| Sr. | Conc. | | Residence time | Temp | Yield of |
|---|---|---|---|---|---|
| No | (M) | Catalyst | (t_{R}) (min)/run | (° C.) | 2a (%) |
| 1^{a} | 0.05 | Fe-zeolite | 17.1/1 | 100 | 10 |
| 2^{a} | 0.05 | Fe-zeolite | 17.1/2 | 120 | 13 |
| 3^{a} | 0.05 | Ru-zeolite | 17.1/2 | 120 | 27 |
| 4^{a} | 0.05 | Ni-zeolite | 17.1/2 | 120 | 48 |
| 5^{a} | 0.05 | Ni-zeolite | 17.1/3 | 120 | 61 |
| 6^{a} | 0.05 | Ni-zeolite | 17.1/4 | 120 | 65 |
| 7^{a} | 0.05 | Ni-zeolite | 17.1/5 | 120 | 65 |
| 8^{a} | 0.1 | Ni-zeolite | 17.1/4 | 120 | 32 |
| 9^{a} | 0.05 | Ni-zeolite | 17.1/4 | 140 | 46 |
| 10^{a} | 0.05 | Ni-zeolite | 17.1/4 | 160 | 20 |
| 11^{b} | 0.05 | Ni-zeolite | 17.1/4 | 120 | 58 |
| 12^{c} | 0.05 | Ni-zeolite | 17.1/4 | 120 | 21 |
| 13^{d} | 0.05 | Ni-zeolite | 17.1/4 | 120 | 45 |
| 14^{a,e} | 0.05 | Ni-zeolite | 27.3/4 | 120 | 68 |

### Example 3: Substrate Scope for Macrocyclic Polyether

To generalize this approach, the substrate scopes for this method were probed with various diol derivatives (FIG. 10, Scheme 3). 0.05 M solution of 1 in dioxane solvent prepared and flowed through 4 mL of the Omnifit column containing Ni-zeolite (1.7 g, 7 cm), 120° C., for residence time 27.3 min, 0.1 mL/min, at 3.3 to 3.5 bar back pressure and 4-time run.

### Example 4: Optimization for Macrolactonization

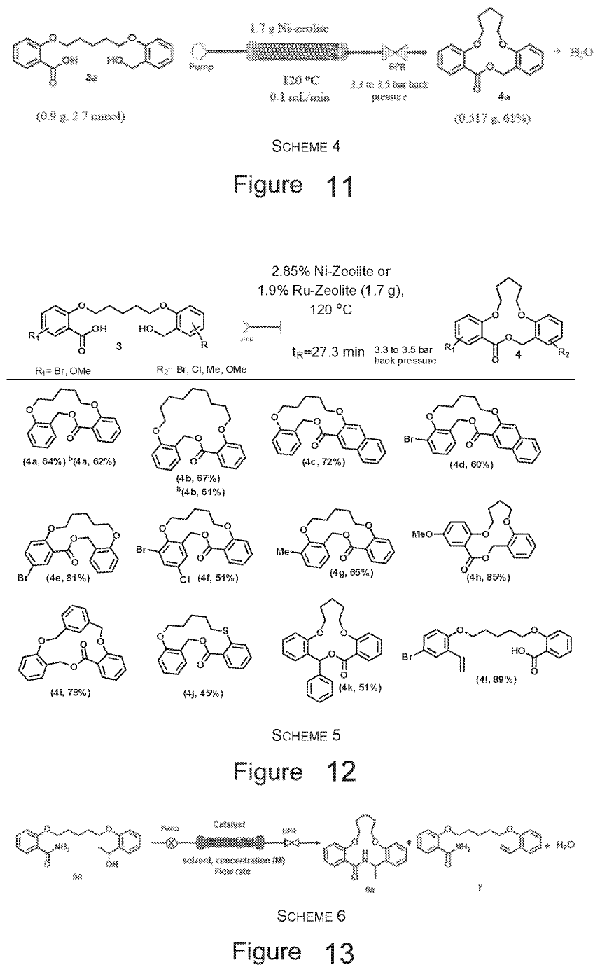

This continuous flow catalytic dehydration is not only limited to the macrocyclic ether, but it has also extended to its application for the formation of macrocyclic lactone. To enable intramolecular macrolactonization, various seco-acids 3 were prepared and subjected to optimization. Initially, this reaction was investigated with seco-acid 3a as a model substrate to provide the macrolactone 4a, by screening different catalysts, solvents, and temperatures in batch and continuous flow conditions. Only β-zeolite in batch and continuous flow conditions afforded 3% and 14% respective yields of macrolactone 4a from seco-acid 3a (FIG. 11, Scheme 4).

Reactions in Batch condition: Compound 3a (0.15 mmol) in solvents (2 mL) and 50 mg of catalyst were stirred in a sealed cap vial at 120° C. for 24 h. The mentioned yields are isolated yields. Reactions in flow conditions: A solution of 3a in (4 mL) solvents was prepared and flowed (0.1 mL/min flow rate) through the Omnifit column (6.6 mm bore×150 mm length) containing the heterogeneous catalysts (1.7 g, 7 cm height), 0.1 mL/min, at 3.3 to 3.5 bar back pressure, at 120° C. The mentioned yields are isolated yields.

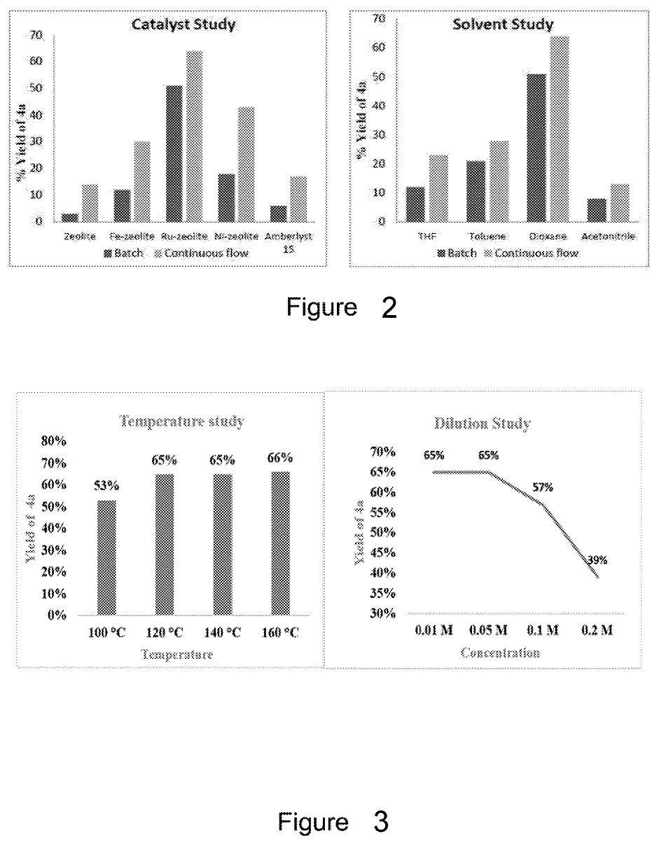

Among various metal-loaded zeolites such as Fe-zeolite, Ru-zeolite, and Ni-zeolite, it was observed that Ru-Zeolite was an effective catalyst for the dehydrative macrolactonization of seco-acid 3a to 4a in 51% and 64% yield under batch and flow conditions (FIG. 2). Amberlyst-15 catalyst delivered less yield of macrocyclic lactone 4a (FIG. 2). Further, in the solvent study, 1,4-dioxane gave the best yield (FIG. 2).

A solution of 3a in dioxane solvents was prepared and flowed (0.1 mL/min flow rate) through the Omnifit column (6.6 mm bore×150 mm length) containing the heterogeneous catalysts (1.7 g, 7 cm height), 3.3 to 3.5 bar back pressure, at different temperatures, and concentration (FIG. 3). The mentioned yields are isolated yields.

The Ni-Zeolite catalyst required four-fold excess time (4 runs) to afford a 66% yield of 3a. Different concentrations for macrolactonization. 0.01 M, 0.05 M, 0.1 M, and 0.2 M concentrations of compound 3a afforded 65%, 65%, 57%, and 39% yields of compound 4a respectively (FIG. 3). In all concentration studies 0.05 M concentration given a maximum yield of compound 4a. Next, temperature study at 100° C., 120° C., 140° C., and 160° C. with the flow rate of 0.1 mL/min afforded a 53%, 65%, 65%, and 66% yield of compound 4a respectively. At 120° C. temperature gives a better yield for macrolactonization 4a (FIG. 3).

### Example 5: General Process for Intramolecular Macrolactonization and Gram Scale for Macrolactonization

In 10-20 mL reagents vials were added 0.05 M solution of seco-acids 3 in dioxane solvent was pumped using a syringe pump through a glass column (Omnifit® (6.6×150 mm) packed bed containing 1.7 g of Ru-zeolite filled up to 7 cm and preheated at 120° C. with the flow rate of 0.1 mL/min at 3.3 to 3.5 bar back pressure was maintained in the reaction. The collected organic layer was concentrated under reduced pressure and the crude product was purified by column chromatography using ethyl acetate:hexane (05:95 to 10:90) as an eluent to afford the compound 4. In addition, this optimized reaction condition was also performed on a large scale (2.7 mmol of 3a) to deliver a 61% yield of compound 4a (FIG. 11, Scheme 4).

### Example 6: Substrate Scope for Macrolactonization

Subsequently, the substrate scope for the macrolactonization with different ring sizes was explored (FIG. 12, Scheme 5). 0.05 M solution of 3 in dioxane (4 mL) solvent prepared and flowed through the Omnifit column (6.6 mm bore×150 mm length) containing Ru-zeolite heterogeneous catalysts (1.7 g, 7 cm), 0.1 mL/min flow rate, at 3.3 to 3.5 bar back pressure, at 120° C. for residence time t_{R}=27.3 min. ^{b}In case of Ni-Zeolite, the reaction mixture flowed for 4 run cycles.

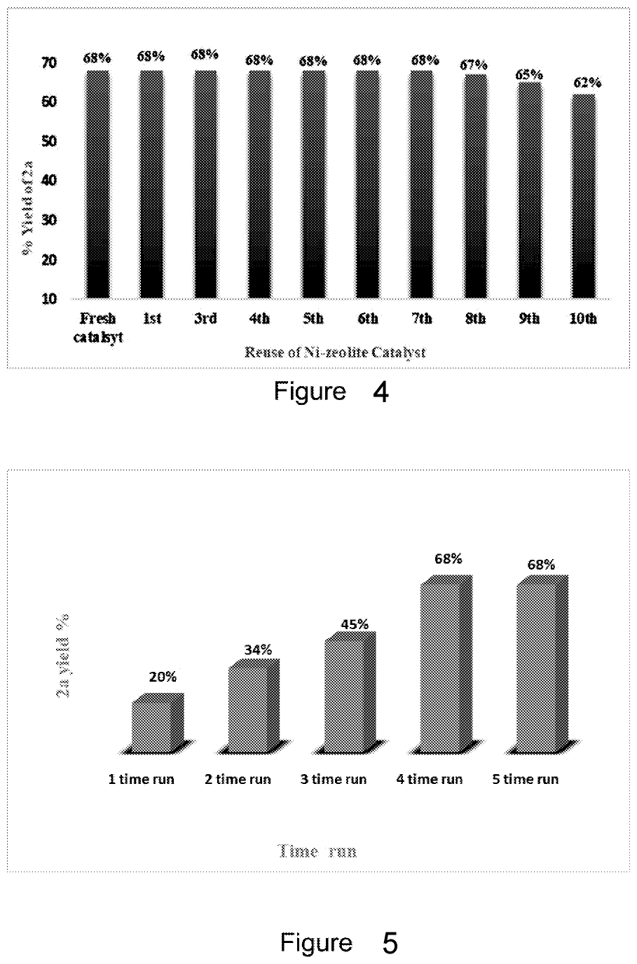

Moreover, Ni-zeolite catalyst is very stable at higher temperatures. This fresh catalyst delivered 68% yield of macrocyclic ether 2a and after one time used the catalyst furnished 68% yield (FIG. 4). The 2nd to 7th time used catalyst afforded the same 68% yield. Moreover, this catalyst was active after the 10th time used (FIG. 4).

### Example 7: Optimization for Macrolactamization

Macrolactams are a very important class of macrocycles found in many drugs and natural products. Thus, they are more significant for the pharmaceutical industry. Despite the importance of macrolactams, there are no reports available for their synthesis under continuous flow conditions using heterogeneous catalysis. Hence, an optimization study was commenced to move a step forward to synthesize macrolactams. Amide 5a (c=0.01 M, 0.08 mmol, 1 equiv.) and catalyst in 8 mL solvent were heated at temperature for 24 h. ^{[b]}48 h. to achieve macrolactam 6a and alkene 7 (FIG. 13, Scheme 6).

After achieving the optimized condition, N-benzylative macrolactamization was optimized under continuous flow conditions. Amide 5a was dissolved in 1,4-dioxane (c=0.01 M) and passed through PTFE tubing with a flow rate of 0.1 mL/min and reacted with Ru-Zeolite catalyst (packed in omnifit column) at 120° C. to afford 68% of the product 6a and traces amount of alkene intermediate 7 (Table 2). The solution of 5a in acetonitrile was flowed through an Omnifit column (6.6 mm bore and 150 mm length) containing fresh 2.43% Ru-Zeolite at 120° C. for time (t_{R}) 28 min (Table 2) (Bed height=8 cm, Catalyst wt.=1.5 gm). ^{[b]}1,4-Dioxane used as a solvent. ^{[c]}10.2 mL/min flow rate. ^{[d]}Bed height=7 cm (1.3 gm catalyst) and 3-4 bar pressure to get the product 6a. Mentioned yields are isolated yields.

TABLE 2
Yield of 6a

| | | | Residence Time | |
|---|---|---|---|---|
| | Conc. of | | t_{R} (min) × | Yield of |
| Entry | 5a (M) | Catalysts | No. of runs | 6a/7 (%) |
| 1 ^{[b]} | 0.01 | 2.43% Ru-Zeolite | 28/1 | 68/trace |
| 2^{[b]} | 0.01 | Fe-Zeolite | 28/1 | 37/41 |
| 3 | 0.01 | 2.43% Ru-Zeolite | 28/1 | 76/trace |
| 4 | 0.01 | β-Zeolite | 28/1 | 41/28 |
| 5 | 0.03 | 2.43% Ru-Zeolite | 28/1 | 74/trace |
| 6 | 0.05 | 2.43% Ru-Zeolite | 28/1 | 76/trace |
| 7 | 0.1 | 2.43% Ru-Zeolite | 28/1 | 50/31 |
| 8 | 0.05 | 2.43% Ru-Zeolite | 28/1 | 77/trace |
| 9^{[c]} | 0.05 | 2.43% Ru-Zeolite | 14/1 | 70/trace |
| 10^{[d]} | 0.05 | 2.43% Ru-Zeolite | 22/1 | 60/25 |

### Example 8: General Process for Macrolactamization and Gram Scale for Macrolactamization

In a 20 mL vial was added amide 5 (0.08 mmol, 1 equiv.), in the presence of acetonitrile (2 mL, 0.05 M) as a solvent. Amides were dissolved by sonication, followed by heating in a water bath to get a clear solution. The resulting reaction mixture was flown using PTFE tubing through the Omnifit® column reactor bed (6.6 mm bore×150 mm length) loaded with 2.43% Ru-Zeolite catalyst and heated at 120° C. (3-4 bar pressure). The progress of the reaction was monitored by the thin-layer chromatography technique. Further, volatile components were evaporated under a vacuum. The residue was directly purified by silica gel chromatography (EtOAc:n-hexane).

In a 250 mL RB flask was added amide 5e (2 gm, 6.06 mmol, 1 equiv.), in the presence of acetonitrile (122 mL, 0.05M) as a solvent. Amides were dissolved by sonication, followed by heating in a water bath to get a clear solution. The resulting reaction mixture was flowed using PTFE tubing through Omnifit® column reactor bed (10 mm bore×150 mm length) loaded with 2.43% Ru-Zeolite catalyst and heated at 120° C. (3-4 bar pressure). The progress of the reaction was monitored by the thin-layer chromatography technique. Further, volatile components were evaporated under a vacuum. The residue was directly purified by silica gel chromatography to afford 61% yield of 6e (1.155 gm) (20% EtOAc in n-hexane). Space time yield (STY)=7.4 gL−1 h⁻¹.

### Example 9: Substrate Scope for Macrolactamization

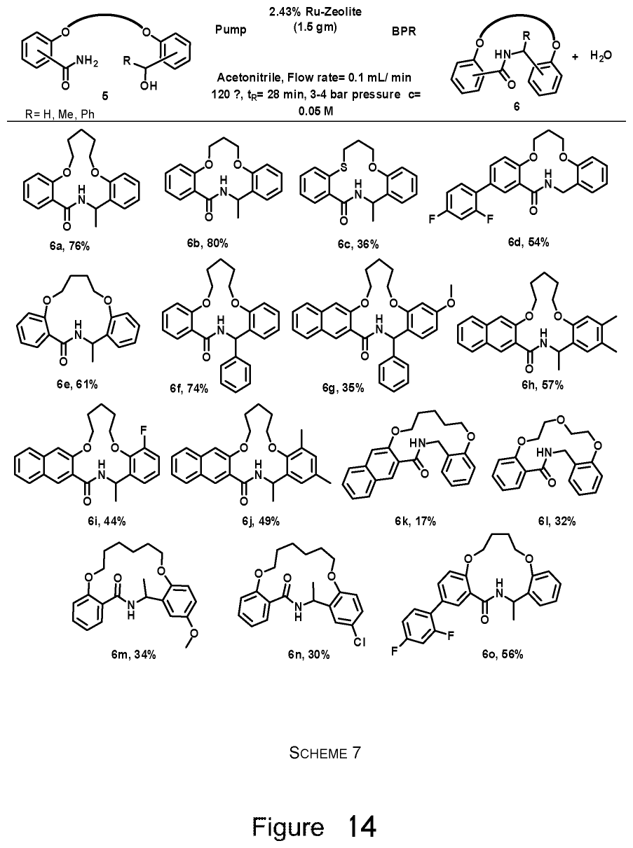

The established optimized reaction condition (Table 2, entry 8) was further utilized for the synthesis of macrolactams (FIG. 14, Scheme 7). The solution of 5 (0.08 mmol, c=0.05 M) in 2 mL acetonitrile flowed through an Omnifit® column (6.6 mm bore and 150 mm length) containing fresh 2.43% Ru-Zeolite (bed height=8 cm, catalyst wt.=1.5 gm) at temperature 120° C. for retention time (t_{R}) 28 min and 3-4 bar pressure to achieve the macrolactams 6. Mentioned yields are isolated yields.

### Example 10: Run Time Study of Ni-Zeolite Catalyst in Continuous Flow

One time run in continuous flow: A 0.05 M solution of the diol 1a in dioxane solvent was pumped using a syringe pump through a glass column (Omnifit® (6.6×150 mm) packed bed and preheated at 120° C. with the flow rate of 0.1 mL/min at 3.3 to 3.5 bar back pressure was maintained in the reaction and residence time t_{R}=23.94 min in one cycle. The collected organic layer was concentrated and further purification afforded a 20% yield of compound 4a (FIG. 5).

Second time run in continuous flow: A 0.05 M solution of the diol 1a in dioxane solvent was pumped using syringe pump through glass column (Omnifit® (6.6×150 mm) packed bed and preheated at 120° C. with the flow rate of 0.1 mL/min at 3.3 to 3.5 bar back pressure was maintained in the reaction and residence time t_{R}=23.94 min in one cycle. After one cycle the same reaction mixture was passed through the same column bed (without activation of catalyst). The collected organic layer was concentrated and further purification afforded a 34% yield of compound 4a (FIG. 5).

Third time run in continuous flow: After two cycles, the same reaction mixture was passed through the same column bed (without activation of catalyst). The collected organic layer was concentrated and further purification afforded a 45% yield of compound 4a (FIG. 5).

Fourth time run in continuous flow: After three cycles the same reaction mixture passed through the same column bed (without activation of catalyst). The collected organic layer was concentrated and further purification affords a 68% yield of compound 4a (FIG. 5).

Fifth time run in continuous flow: After four cycles the same reaction mixture passed through the same column bed (without activation of catalyst). The collected organic layer was concentrated and further purification affords a 68% yield of compound 4a (FIG. 5).

### Example 11: Analytical Data of Products

7,8,9,10-tetrahydro-6H,16H,18H-dibenzo[b,g][1,5,9]trioxacyclotetradecine (2a): A 0.05 M solution of 1a ((pentane-1,5-diylbis(oxy))bis(2,1-phenylene))dimethanol (63 mg, 0.199 mmol) in (4 mL) dioxane solvent was pumped using syringe pump through glass column (Omnifit® (6.6×150 mm) packed bed containing 1.7 g of Ni-zeolite filled up to 7 cm and preheated at 120° C. with the flow rate of 0.1 mL/min at 3.3 to 3.5 bar back pressure was maintained in the reaction and residence time t_{R}=27.3 min. After a four-time run. The collected organic layer was concentrated under reduced pressure and the crude product was purified by column chromatography using ethyl acetate:hexane (5:95) as an eluent to afford the compound 2a (40.3 mg, 68%) as a white solid, weight hourly space velocity (WHSV)=56 h⁻¹, space-time yield=17.57 g L⁻¹ h⁻¹. Melting point: 80-83° C. Large scale reaction for the synthesis of macrocyclic ether 2a: A 0.05 M solution of 1a ((pentane-1,5-diylbis(oxy))bis(2,1-phenylene))dimethanol (2 g, 6.32 mmol) in dioxane solvent was pumped using a syringe pump through two glass column (Omnifit® (6.6×150 mm) packed bed containing 1.7 g of Ni-zeolite filled up to 7 cm each column bed and preheated at 120° C. with the flow rate of 0.1 mL/min at 4-5 bar back pressure was maintained in the reaction and residence time t_{R}=27.3 min each column bed. After a two-time run, the collected organic layer was concentrated under reduced pressure, and the crude product was purified by column chromatography using ethyl acetate:hexane (5:95) as an eluent to afford the compound 2a (1.231 g, 4.12 mmol, 65%) as a white solid, weight hourly space velocity (WHSV)=56 h⁻¹, space-time yield=17.57 g L⁻¹ h⁻¹. ¹H NMR (400 MHz, CDCl₃) δ 7.47 (dd, J=7.4, 1.7 Hz, 2H), 7.41-7.36 (m, 2H), 7.03 (t, J=7.4 Hz, 2H), 6.98 (d, J=8.1 Hz, 2H), 4.75 (s, 4H), 4.21 (t, J=5.1 Hz, 4H), 2.08-2.01 (m, 2H), 1.98 (dd, J=11.4, 5.9 Hz, 4H); ¹³C{¹H} NMR (100 MHz, CDCl₃) δ 158.2, 131.9, 129.6, 126.7, 120.2, 111.9, 69.3, 66.9, 28.0, 21.4; FTIR (neat): 2931, 2864, 1596, 1246 cm⁻¹; HRMS (ESI-TOF) m/z; calculated for C₁₉H₂₂O₃Na, [M+Na]⁺ 321.1466, found 321.1469. Crystal preparation for 2a: The crystals are grown by a simple recrystallization method where pure compounds isolated after column chromatography are dissolved in dichloromethane and layered with hexane, kept at room temperature to get compound 2a pure crystal.

7,8-dihydro-6H,14H,16H-dibenzo[b,g][1,5,9]trioxacyclododecine (2b): (31.3 mg, 58%) as a white solid, weight hourly space velocity (WHSV)=45 h⁻¹, space-time yield=11.64 g L⁻¹ h⁻¹. Melting point: 65-70° C.; ¹H NMR (400 MHz, CDCl₃) δ 7.28 (dd, J=7.7, 1.3 Hz, 2H), 7.26-7.22 (m, 2H), 6.92-6.83 (m, 4H), 4.60 (s, 4H), 4.28-4.22 (m, 4H), 2.31-2.25 (m, 2H); ¹³C{¹H} NMR (100 MHz, CDCl₃) δ 158.2, 131.3, 129.6, 127.3, 120.3, 111.6, 69.4, 68.1, 28.5; FTIR (neat): 2930, 2860, 1596, 1246 cm⁻¹; HRMS (ESI-TOF) m/z; calculated for C₁₇H₁₈O₃Na, [M+Na]⁺ 293.1153, found 293.1154.

6,7,8,9-tetrahydro-15H,17H-dibenzo[b,g][1,5,9]trioxacyclotridecine (2c): (32 mg, 56%) as a white solid, weight hourly space velocity (WHSV)=47 h⁻¹, space-time yield=11.50 g L⁻¹ h⁻¹ and starting material recovered 1c (25 mg, 41%), as semisolid; ¹H NMR (400 MHz, CDCl₃) δ 7.33 (dd, J=7.8, 1.7 Hz, 2H), 7.28-7.23 (m, 2H), 6.95 (dd, J=10.7, 4.4 Hz, 4H), 4.63 (s, 4H), 4.14 (t, J=7.4, 3.2 Hz, 4H), 2.07 (td, J=5.4, 2.6 Hz, 4H); ¹³C{¹H} NMR (100 MHz, CDCl₃) δ 158.7, 131.8, 129.8, 128.7, 121.7, 115.9, 70.7, 69.3, 27.9. FTIR (neat): 2926, 2856, 1455, 1240 cm⁻¹; HRMS (ESI-TOF) m/z; calculated for C₁₈H₂₀O₃Na, [M+Na]⁺ 307.1309, found 307.1305.

6,7,8,9,10,11-hexahydro-17H,19H-dibenzo[b,g][1,5,9]trioxacyclopentadecine (2d): (34.3 mg, 61%) as a white solid, weight hourly space velocity (WHSV)=52 h⁻¹, space-time yield=17.9 g L¹ h⁻¹ and starting material recovered 1d (12.5 mg, 21%). Melting point: 66-68° C.; ¹H NMR (400 MHz, CDCl₃) δ 7.40 (dd, J=7.4, 1.7 Hz, 2H), 7.25-7.19 (m, 2H), 6.92 (td, J=7.4, 1.0 Hz, 2H), 6.85 (d, J=8.2 Hz, 2H), 4.66 (s, 4H), 4.06 (t, 4H), 1.83 (dd, J=9.9, 5.3 Hz, 4H), 1.76-1.69 (m, 4H); ¹³C{¹H} NMR (100 MHz, CDCl₃) δ 157.1, 130.7, 129.0, 127.6, 120.6, 111.61, 68.4, 68.1, 28.9, 27.2; FTIR (neat): 2925, 2854, 1453, 1239 cm⁻¹; HRMS (ESI-TOF) m/z; calculated for C₂₀H₂₄O₃Na, [M+Na]⁺ 335.1622, found: 335.1624.

6,7,8,9,10,11,12,13-octahydro-19H,21H-dibenzo[b,g][1,5,9]trioxacycloheptadecine (2e): (34.6 mg, 51%) as a white solid, weight hourly space velocity (WHSV)=52 h⁻¹, space-time yield=11.31 g L⁻¹ h⁻¹ and starting material recovered 1e (10 mg, 14%). Melting point: 60-63° C.; ¹H NMR (400 MHz, CDCl₃) δ 7.46 (d, J=7.4 Hz, 2H), 7.26-7.19 (m, 2H), 6.95 (t, J=7.4 Hz, 2H), 6.85 (d, J=8.2 Hz, 2H), 4.71 (s, 4H), 4.06-4.00 (m, 4H), 1.84-1.76 (m, 4H), 1.65 (dd, J=14.5, 7.0 Hz, 4H), 1.48 (dd, J=8.5, 5.2 Hz, 4H); ¹³C{¹H} NMR (100 MHz, CDCl₃) δ 156.8, 129.6, 128.6, 127.6, 120.4, 111.0, 67.7, 67.6, 28.9, 27.7, 25.2; FTIR (neat): 2930, 2859, 1455, 1286 cm⁻¹; HRMS (ESI-TOF) m/z; calculated for C₂₂H₂₉O₃, [M+H]⁺ 341.2116, found 341.2115.

7,8,9,10,11,12,13,14-octahydro-6H,20H,22H-dibenzo[b,g][1,5,9]trioxacyclooctadecine (2f): (26.2 mg, 37%) as a white solid, weight hourly space velocity (WHSV)=59 h⁻¹, space-time yield=6.21 g L⁻¹ h⁻¹, and starting material recovered if (9.5 mg, 13%), as a semisolid; ¹H NMR (400 MHz, CDCl₃) δ 7.46 (dd, J=7.5, 1.6 Hz, 2H), 7.23 (td, J=8.1, 1.8 Hz, 2H), 6.96 (td, J=7.4, 0.9 Hz, 2H), 6.86 (d, J=8.2 Hz, 2H), 4.70 (s, 4H), 4.02 (t, 4H), 1.84-1.77 (m, 4H), 1.63-1.55 (m, 4H), 1.47 (ddd, J=12.0, 8.9, 4.7 Hz, 6H); ¹³C{¹H} NMR (100 MHz, CDCl₃) δ 156.9, 129.5, 128.6, 127.5, 120.5, 111.3, 68.4, 67.3, 29.4, 29.1, 28.7, 26.7; FTIR (neat): 2931, 2858, 1455, 1240 cm⁻¹; HRMS (ESI-TOF) m/z; calculated for C₂₃H₃₀O₃Na, [M+Na]⁺ 377.2092, found: 377.2087.

6,7,8,9,10,11,12,13,14,15-decahydro-21H,23H-dibenzo[b,g][1,5,9]trioxacyclononadecine (2g): (27.4 mg, 39%) as a semi solid, weight hourly space velocity (WHSV)=62 h⁻¹, space-time yield=6.90 g L⁻¹ h⁻¹, and starting material recovered 1g (14.3 mg, 19%); ¹H NMR (400 MHz, CDCl₃) δ 7.51-7.47 (m, 2H), 7.26-7.20 (m, 2H), 6.99-6.93 (m, 2H), 6.85 (d, J=8.0 Hz, 2H), 4.72 (s, 4H), 4.01 (t, J=5.3 Hz, 4H), 1.78 (dt, J=10.7, 5.9 Hz, 4H), 1.57 (d, J=3.8 Hz, 4H), 1.40 (d, J=8.5 Hz, 8H); ¹³C{¹H} NMR (100 MHz, CDCl₃) δ 156.7, 128.4, 128.4, 127.6, 120.4, 111.2, 67.9, 67.8, 28.9, 28.0, 27.9, 25.9; FTIR (neat): 2925, 2854, 1453, 1239 cm⁻¹; HRMS (ESI-TOF) m/z; calculated for C₂₄H₃₂O₃Na, [M+Na]⁺ 391.2249 found 391.2263.

3-bromo-7,8,9,10-tetrahydro-6H,16H,18H-dibenzo[b,g][1,5,9]trioxacyclotetradecine (2h): (52.1 mg, 69%) as a white solid, weight hourly space velocity (WHSV)=64 h⁻¹, space-time yield=23.04 g L⁻¹ h⁻¹, and starting material recovered 1h (8.5 mg, 11%). Melting point: 139-142° C.; ¹H NMR (400 MHz, CDCl₃) δ 7.44 (d, J=2.5 Hz, 1H), 7.31 (ddd, J=6.2, 4.7, 2.1 Hz, 2H), 7.23 (dd, J=7.8, 1.5 Hz, 1H), 6.88 (td, J=7.4, 1.0 Hz, 1H), 6.83 (d, J=8.2 Hz, 1H), 6.71 (d, J=8.7 Hz, 1H), 4.58 (s, 2H), 4.54 (s, 2H), 4.04 (dd, J=10.3, 5.5 Hz, 4H), 1.91-1.77 (m, 6H); ¹³C{¹H} NMR (100 MHz, CDCl₃) δ 158.2, 157.3, 134.5, 132.2, 132.0, 129.8, 128.9, 126.4, 120.3, 113.8, 112.3, 111.9, 69.5, 68.6, 67.4, 66.8, 27.9, 27.9, 21.3; FTIR (neat): 2925, 2866, 1456, 1270 cm⁻¹; HRMS (ESI-TOF) m/z; calculated for C₁₉H₂₁BrO₃Na, [M+Na]⁺ 399.0572, found 399.0568.

3-chloro-7,8,9,10-tetrahydro-6H,16H,18H-dibenzo[b,g][1,5,9]trioxacyclotetradecine (2i): (27.6 mg, 55%) as a white solid, weight hourly space velocity (WHSV)=56 h⁻¹, space-time yield=12.97 g L⁻¹ h⁻¹, and starting material recovered 1i (17.8 mg, 32%). Semisolid. ¹H NMR (400 MHz, CDCl₃) δ 7.35 (dd, J=7.4, 1.7 Hz, 1H), 7.31-7.25 (m, 2H), 6.96-6.87 (m, 3H), 6.86 (d, J=1.9 Hz, 1H), 4.61 (s, 2H), 4.58 (s, 2H), 4.08 (dt, J=6.8, 5.1 Hz, 4H), 1.99-1.80 (m, 6H); ¹³C{¹H} NMR (100 MHz, CDCl₃) δ 158.8, 158.2, 134.9, 132.6, 131.9, 129.8, 126.4, 125.3, 120.3, 120.2, 112.7, 111.9, 69.4, 68.7, 67.2, 66.7, 27.9, 27.8, 21.2. FTIR (neat): 2934, 2871, 1503, 1272 cm⁻¹; HRMS (ESI-TOF) m/z; calculated for C₁₉H₂₁O₃ClNa, [M+Na]⁺ 355.1078, found 355.1072.

2-nitro-7,8,9,10-tetrahydro-6H,16H,18H-dibenzo[b,g][1,5,9]trioxacyclotetradecine (2j): (25.4 mg, 49%) as a white solid, weight hourly space velocity (WHSV)=59 h⁻¹, space-time yield=10.64 g L⁻¹ h⁻¹, and starting material recovered 1j (9.8 mg, 18%). Melting point: 157-158° C.; ¹H NMR (400 MHz, CDCl₃) δ 8.25 (d, J=2.8 Hz, 1H), 8.17 (dd, J=9.0, 2.8 Hz, 1H), 7.32 (dd, J=7.4, 1.7 Hz, 1H), 7.28-7.21 (m, 1H), 6.89 (ddd, J=11.8, 6.4, 2.7 Hz, 2H), 6.83 (d, J=8.2 Hz, 1H), 4.61 (s, 2H), 4.61 (s, 2H), 4.15 (t, J=4.7 Hz, 2H), 4.06 (t, J=5.0 Hz, 2H), 1.92-1.80 (m, 6H); ¹³C{¹H} NMR (100 MHz, CDCl₃) δ 163.4, 158.1, 140.8, 132.1, 130.1, 127.7, 127.5, 126.2, 125.9, 120.4, 111.87, 111.4, 69.7, 68.7, 67.9, 66.4, 27.7, 27.5, 21.0; FTIR (neat): 2934, 2871, 1503, 1272 cm⁻¹; HRMS (ESI-TOF) m/z; calculated for C₁₉H₂₂NO₅, [M+H]⁺ 344.1499, found 344.1502.

2,6,9-trioxa-1,7(1,2),4(1,3)-tribenzenacyclodecaphane (2k): (35.7 mg, 54%) as a white solid, weight hourly space velocity (WHSV)=56 h⁻¹, space-time yield=12.36 g L⁻¹ h⁻¹, Melting point: 143-145° C.; ¹H NMR (400 MHz, CDCl₃) δ 8.19 (s, 1H), 7.38 (dd, J=7.3, 1.6 Hz, 2H), 7.24 (td, J=7.9, 1.8 Hz, 3H), 7.13 (d, J=7.5 Hz, 2H), 6.91 (dd, J=11.7, 5.3 Hz, 4H), 5.20 (s, 4H), 4.67 (s, 4H); ¹³C{¹H} NMR (100 MHz, CDCl₃) δ 157.8, 138.6, 132.5, 129.9, 127.7, 126.7, 126.2, 124.8, 120.8, 112.8, 69.3, 69.1; FTIR (neat): 2913, 2878, 1495, 1254 cm⁻¹; HRMS (ESI-TOF) m/z; calculated for C₂₂H₂₀O₃Na, [M+Na]⁺ 355.1310, found 355.1316.

6,7,10,11-tetrahydro-9H,17H,19H-dibenzo[e,j][1,4,8,12]tetraoxacyclopentadecine (2l): (35.7 mg, 59%) as a white solid, weight hourly space velocity (WHSV)=50 h⁻¹, space-time yield=13.50 g L⁻¹ h⁻¹, Melting point: 112-114° C.; ¹H NMR (400 MHz, CDCl₃) δ 7.40-7.35 (m, 2H), 7.27-7.20 (m, 2H), 6.94 (t, J=7.4 Hz, 2H), 6.85 (d, J=8.2 Hz, 2H), 4.68 (s, 4H), 4.20-4.14 (m, 4H), 3.92-3.87 (m, 4H); ¹³C{¹H} NMR (100 MHz, CDCl₃) δ 157.5, 131.3, 129.3, 127.8, 120.9, 112.4, 69.9, 68.5, 67.9; FTIR (neat): 2871, 1492, 1249 cm⁻¹; HRMS (ESI-TOF) m/z; calculated for C₁₈H₂₀O₄Na, [M+Na]⁺ 323.1259, found 323.1253.

6,7,9,10,12,13-hexahydro-19H,21H-dibenzo[k,p][1,4,7,10,14]pentaoxacycloheptadecine (2m): (30.5 mg, 44%) as a semisolid, weight hourly space velocity (WHSV)=59 h⁻¹, space-time yield=8.60 g L⁻¹ h⁻¹, ¹H NMR (400 MHz, CDCl₃) δ 7.42 (dd, J=7.5, 1.6 Hz, 2H), 7.23 (td, J=8.0, 1.8 Hz, 2H), 6.96 (td, J=7.4, 0.9 Hz, 2H), 6.85 (d, J=8.2 Hz, 2H), 4.73 (s, 4H), 4.17-4.13 (m, 4H), 3.92-3.89 (m, 4H), 3.78 (s, 4H); ¹³C{¹H} NMR (100 MHz, CDCl₃) δ 156.7, 130.1, 128.7, 128.1, 121.0, 111.7, 70.8, 69.8, 68.1, 67.5; FTIR (neat): 2912, 2871, 1451, 1243 cm⁻¹; HRMS (ESI-TOF) m/z; calculated for C₂₀H₂₄O₅Na, [M+Na]⁺ 367.1521, found 367.1515.

6,7,9,10,12,13,15,16-octahydro-22H,24H-dibenzo[n,s][1,4,7,10,13,17]hexaoxacycloicosine (2n): (36.4 mg, 47%) as a white solid, weight hourly space velocity (WHSV)=66 h⁻¹, space-time yield=10.97 g L⁻¹ h⁻¹, Melting point: 90-92° C.; ¹H NMR (400 MHz, CDCl₃) δ 7.48 (dd, J=7.4, 1.6 Hz, 2H), 7.23 (td, J=7.9, 1.7 Hz, 2H), 7.01-6.95 (m, 2H), 6.88-6.83 (m, 2H), 4.73 (s, 4H), 4.19-4.12 (m, 4H), 3.88-3.84 (m, 4H), 3.82-3.76 (m, 4H), 3.76-3.70 (m, 4H); ¹³C{¹H} NMR (100 MHz, CDCl₃) δ 156.4, 128.9, 128.5, 128.0, 121.1, 111.8, 71.5, 70.8, 69.8, 68.6, 67.6; FTIR (neat): 2869, 1452, 1244 cm⁻¹; HRMS (ESI-TOF) m/z; calculated for C₂₂H₂₈O₆Na, [M+Na]⁺ 411.1783, found: 411.1779.

7,8,16,17-tetrahydro-6H,14H-dibenzo[c,j][1,5,9]trioxacyclotridecine (2o): (34.10 mg, 60%) as a white solid, weight hourly space velocity (WHSV)=47 h⁻¹, space-time yield=13.12 g L⁻¹ h⁻¹, melting point: 61-65° C.; ¹H NMR (400 MHz, CDCl₃) δ 7.43-7.37 (m, 1H), 7.30-7.23 (m, 1H), 7.16 (t, J=8.2 Hz, 2H), 6.96 (t, J=7.5 Hz, 1H), 6.89-6.82 (m, 2H), 6.79 (d, J=8.0 Hz, 1H), 4.71 (s, 2H), 4.22-4.14 (m, 4H), 3.61-3.54 (m, 2H), 3.03 (dd, J=9.3, 6.7 Hz, 2H), 2.36 (dt, J=9.6, 4.8 Hz, 2H); ¹³C{¹H} NMR (100 MHz, CDCl₃) δ 157.6, 157.2, 132.7, 131.1, 129.5, 127.6, 127.3, 127.0, 120.8, 120.6, 110.8, 110.4, 68.3, 68.1, 67.9, 63.8, 30.6, 29.4; FTIR (neat): 2929, 2865, 1455, 1240 cm⁻¹; HRMS (ESI-TOF) m/z; calculated for C₁₈H₂₀O₃Na, [M+Na]⁺307.1309, found 307.1307.

6,7,8,9,17,18-hexahydro-15H-dibenzo[b,h][1,5,1]trioxacyclotetradecine (2p): (39.8 mg, 67%) as a semisolid, weight hourly space velocity (WHSV)=53 h⁻¹, space-time yield=17.10 g L⁻¹ h⁻¹, ¹H NMR (400 MHz, CDCl₃) δ 7.35 (dd, J=7.3, 1.6 Hz, 1H), 7.30-7.24 (m, 1H), 7.18 (td, J=7.9, 1.7 Hz, 1H), 7.14 (dd, J=7.4, 1.5 Hz, 1H), 6.95 (dd, J=12.0, 4.6 Hz, 2H), 6.88 (dd, J=7.4, 0.9 Hz, 1H), 6.82 (d, J=8.1 Hz, 1H), 4.64 (s, 2H), 4.16 (t, J=5.2 Hz, 2H), 4.05 (t, J=4.9 Hz, 2H), 3.86-3.79 (m, 2H), 2.99-2.91 (m, 2H), 2.13-2.07 (m, 4H); ¹³C{¹H} NMR (100 MHz, CDCl₃) δ 157.9, 157.6, 130.9, 130.8, 129.4, 128.7, 127.7, 127.4, 121.1, 120.6, 113.7, 111.8, 71.4, 68.9, 68.6, 67.9, 32.5, 27.1, 26.3. FTIR (neat): 2928, 2861, 1455 cm⁻¹; HRMS (ESI-TOF) m/z; calculated for C₁₉H₂₂O₃Na, [M+Na]⁺ 321.1466, found 321.1459.

7,8,9,10,18,19-hexahydro-6H,16H-dibenzo[b,h][1,5,10]trioxacyclopentadecine (2q): (39.8 mg, 64%) as a semisolid, weight hourly space velocity (WHSV)=52 h⁻¹, space-time yield=16.33 g L⁻¹ h⁻¹, and starting material recovered 1q (7.3 mg, 11%). ¹H NMR (400 MHz, CDCl₃) δ 7.37 (dd, J=7.4, 1.6 Hz, 1H), 7.27 (ddd, J=6.8, 5.7, 1.7 Hz, 1H), 7.18 (ddd, J=7.3, 6.7, 2.6 Hz, 2H), 6.97-6.83 (m, 4H), 4.63 (s, 2H), 4.11 (t, J=5.1 Hz, 2H), 4.03 (t, 2H), 3.77-3.70 (m, 2H), 3.04-2.96 (m, 2H), 1.96 (d, J=7.3 Hz, 2H), 1.87 (ddd, J=12.0, 8.8, 4.8 Hz, 4H); ¹³C{¹H} NMR (100 MHz, CDCl₃) δ 157.5, 157.3, 131.0, 130.5, 129.2, 127.7, 127.6, 127.3, 120.6, 120.5, 111.8, 111.8, 71.16, 67.8, 67.1, 67.0, 32.1, 28.9, 28.8, 22.7; FTIR (neat): 2929, 2865, 1455, 1240 cm⁻¹; FIRMS (ESI-TOF) m/z; calculated for C₂₀H₂₄O₃Na, [M+Na]⁺ 335.1622, found 335.1620.

6,7,8,9,10,11,19,20-octahydro-17H-dibenzo[b,h][1,5,10]trioxacyclohexadecine (2r): (42.1 mg, 66%) as a semisolid, weight hourly space velocity (WHSV)=55 h⁻¹, space-time yield=17.81 g L⁻¹ h⁻¹, and starting material recovered 1r (5.8 mg, 8%). ¹H NMR (400 MHz, CDCl₃) δ 7.37 (d, J=7.4 Hz, 1H), 7.31-7.24 (m, 1H), 7.22-7.15 (m, 2H), 6.95 (t, J=7.4 Hz, 1H), 6.89 (t, J=7.2 Hz, 2H), 6.84 (d, J=8.1 Hz, 1H), 4.60 (s, 2H), 4.11-4.05 (m, 2H), 4.05-3.96 (m, 2H), 3.75-3.66 (m, 2H), 3.05-2.95 (m, 2H), 1.93-1.83 (m, 4H), 1.78 (dd, J=6.0, 2.9 Hz, 4H). ¹³C{¹H} NMR (100 MHz, CDCl₃) δ 157.6, 157.4, 130.8, 130.3, 129.2, 127.7, 126.9, 126.8, 120.4, 120.3, 111.3, 110.8, 70.9, 68.8, 68.6, 68.4, 31.5, 29.7, 29.6, 28.5, 28.4; FTIR (neat): 2925, 2859, 1456, 1238 cm⁻¹; HRMS (ESI-TOF) m/z; calculated for C₂₁H₂₇O₃, [M+H]⁺ 327.1960, found: 327.1956.

6,7,8,9,10,11,20,21-octahydro-17H,19H-dibenzo[b,i][1,5,11]trioxacycloheptadecine (2s): (41.7 mg, 61%) as a white solid, weight hourly space velocity (WHSV)=57 h⁻¹, space-time yield=16.31 g L⁻¹ h⁻¹, and starting material recovered is (12.2 mg, 17%). Melting point: 62-64° C.; ¹H NMR (400 MHz, CDCl₃) δ 7.33 (dd, J=7.4, 1.6 Hz, 1H), 7.29-7.23 (m, 1H), 7.18-7.09 (m, 2H), 6.94 (td, J=7.4, 0.9 Hz, 1H), 6.91-6.82 (m, 3H), 4.55 (s, 2H), 4.09-3.99 (m, 4H), 3.63 (t, J=6.6 Hz, 2H), 2.71-2.63 (m, 2H), 1.97-1.89 (m, 2H), 1.89-1.80 (m, 4H), 1.70 (dd, J=6.4, 3.4 Hz, 4H); ¹³C{¹H} NMR (100 MHz, CDCl₃) δ 157.6, 157.1, 131.3, 130.6, 130.0, 129.1, 127.6, 127.0, 120.6, 120.4, 112.4, 111.4, 70.4, 68.9, 68.0, 67.7, 30.0, 29.7, 29.6, 27.9, 27.3, 27.1; FTIR (neat): 2930, 2859, 1491, 1240 cm⁻¹; FIRMS (ESI-TOF) m/z; calculated for C₂₂H₂₉O₃, [M+H]⁺ 341.2116, found 341.2114.

7,8,9,10,11,12,13,14,22,23-decahydro-6H,20H dibenzo[b,h][1,5,10]trioxacyclononadecine (2t): (30.3 mg, 41%) as a semisolid, weight hourly space velocity (WHSV)=62 h⁻¹, space-time yield=7.96 g L⁻¹ h⁻¹, ¹H NMR (400 MHz, CDCl₃) δ 7.43 (dd, J=7.5, 1.5 Hz, 1H), 7.26-7.22 (m, 1H), 7.22-7.16 (m, 2H), 6.95 (td, J=7.4, 0.8 Hz, 1H), 6.89 (td, J=7.5, 1.0 Hz, 1H), 6.84 (dd, J=11.2, 3.9 Hz, 2H), 4.65 (s, 2H), 4.05-4.01 (m, 2H), 4.01-3.97 (m, 2H), 3.84-3.77 (m, 2H), 3.10-3.02 (m, 2H), 1.86-1.78 (m, 4H), 1.62-1.53 (m, 4H), 1.51-1.42 (m, 6H); ¹³C{¹H} NMR (100 MHz, CDCl₃) δ 157.4, 156.7, 130.9, 129.0, 128.6, 127.6, 127.2, 126.8, 120.4, 120.3, 111.1, 110.9, 70.9, 68.5, 67.9, 67.7, 31.6, 29.8, 29.2, 28.7, 28.4, 27.2, 26.9. FTIR (neat): 2926, 2856, 1492, 1239 cm⁻¹; HRMS (ESI-TOF) m/z; calculated for C₂₄H₃₂O₃Na, [M+Na]⁺ 391.5249, found: 391.5240.

7,8,9,10-tetrahydro-6H,16H,18H-dibenzo[b,g][1,5,9]trioxacyclotetradecin-16-one (4a): A 0.05 M solution of 3a 2-((5-(2-(hydroxymethyl)phenoxy)pentyl)oxy)benzoic acid (66 mg, 0.199 mmol) in (4 mL) dioxane solvent was pumped using syringe pump through glass column (Omnifit® (6.6×150 mm) packed bed containing 1.7 g of Ru-zeolite filled up to 7 cm and preheated at 120° C. with the flow rate of 0.1 mL/min at 3.3 to 3.5 bar back pressure was maintained in the reaction. The collected organic layer was concentrated under reduced pressure and the crude product was purified by column chromatography using ethyl acetate:hexane (10:90) as an eluent to afford the compound 4a (39.9 mg, 64%) as a white solid, weight hourly space velocity (WHSV)=52 h⁻¹, space-time yield=16.37 g L⁻¹ h⁻¹, Melting point: 86-89° C.

Large scale reaction for macrocyclic ether: A 0.05 M solution of 3a 2-((5-(2-(hydroxymethyl)phenoxy)pentyl)oxy)benzoic acid (0.9 g, 2.72 mmol) in dioxane solvent was pumped using a syringe pump through two glass column (Omnifit® (6.6×150 mm) packed bed containing 1.7 g of Ru-zeolite filled up to 7 cm each column bed and preheated at 120° C. with the flow rate of 0.1 mL/min at 4-5 bar back pressure was maintained in the reaction and residence time t_{R}=27.3 min each column bed. After a two-time run, the collected organic layer was concentrated under reduced pressure, and the crude product was purified by column chromatography using ethyl acetate:hexane (5:95) as an eluent to afford the compound 4a (0.517 g, 1.67 mmol, 61%) as a white solid, weight hourly space velocity (WHSV)=52 h⁻¹, space-time yield=15.98 g L⁻¹ h⁻¹, ¹H NMR (400 MHz, CDCl₃) δ 7.77 (dd, J=7.7, 1.8 Hz, 1H), 7.44 (dd, J=7.3, 1.7 Hz, 1H), 7.38 (ddd, J=8.4, 7.4, 1.8 Hz, 1H), 7.31 (td, J=8.0, 1.8 Hz, 1H), 6.96-6.92 (m, 1H), 6.90 (ddd, J=8.2, 3.3, 2.1 Hz, 2H), 6.84 (d, J=8.3 Hz, 1H), 5.39 (s, 2H), 4.13 (t, J=5.3 Hz, 2H), 4.01-3.91 (m, 2H), 1.92 (tt, J=11.0, 5.6 Hz, 4H), 1.74 (td, J=10.4, 5.5 Hz, 2H); ¹³C{¹H} NMR (100 MHz, CDCl₃) δ 168.5, 158.6, 158.2, 133.1, 131.9, 130.6, 123.9, 121.3, 120.2, 120.1, 112.6, 111.4, 67.8, 64.9, 63.6, 27.2, 26.6, 20.5. FTIR (neat): 2941, 1691, 1601, 1240 cm⁻¹; HRMS (ESI-TOF) m/z; calculated for C₁₉H₂₀O₄Na, [M+Na]⁺ 335.1259, found 335.1253.

7,8,9,10,11,12,13,14-octahydro-6H,20H,22H-dibenzo[b,g][1,5,9]trioxacyclooctadecin-20-one (4b): (37.1 mg, 67%) as a white solid, weight hourly space velocity (WHSV)=62 h⁻¹, space-time yield=21.25 g L⁻¹ h⁻¹, ¹H NMR (400 MHz, CDCl₃) δ 7.70 (dd, J=7.6, 1.7 Hz, 1H), 7.45 (dd, J=7.5, 1.6 Hz, 1H), 7.42-7.36 (m, 1H), 7.32-7.27 (m, 1H), 6.97 (dd, J=10.9, 4.0 Hz, 1H), 6.90 (dd, J=16.0, 8.3 Hz, 3H), 5.44 (s, 2H), 4.09-4.04 (m, 2H), 4.04-3.99 (m, 2H), 1.86-1.73 (m, 4H), 1.54 (dt, J=11.1, 6.6 Hz, 4H), 1.49-1.41 (m, 4H), 1.38 (dd, J=11.6, 5.6 Hz, 2H). ¹³C{¹H} NMR (100 MHz, CDCl₃) δ 166.2, 158.8, 157.2, 133.1, 131.4, 129.9, 129.4, 125.1, 121.1, 120.3, 119.9, 113.1, 111.6, 68.2, 68.0, 62.0, 28.7, 28.0, 27.9, 27.6, 27.5, 25.6, 25.2. FTIR (neat):1717, 1600, 1295, 1126 cm⁻¹; HRMS (ESI-TOF) m/z; calculated for C₂₃H₂₈O₄Na, [M+Na]⁺ 391.1885, found 391.1884.

7,8,9,10-tetrahydro-6H,18H,20H-benzo[b]naphtho[2,3-g][1,5,9]trioxacyclotetradecin-18-one (4c): (52.1 mg, 72%) as a white solid. weight hourly space velocity (WHSV)=61 h⁻¹, space-time yield=24.05 g L⁻¹ h⁻¹, ¹H NMR (400 MHz, CDCl₃) δ 8.27 (s, 1H), 7.80 (d, J=8.1 Hz, 1H), 7.67 (d, J=8.2 Hz, 1H), 7.47 (ddd, J=7.9, 4.4, 1.2 Hz, 2H), 7.36-7.29 (m, 2H), 7.08 (s, 1H), 6.96-6.87 (m, 2H), 5.44 (s, 2H), 4.14 (t, J=5.4 Hz, 2H), 4.11-4.05 (m, 2H), 2.01-1.88 (m, 4H), 1.81 (td, J=10.5, 5.5 Hz, 2H); ¹³C{¹H} NMR (100 MHz, CDCl₃) δ 168.5, 158.6, 155.0, 136.1, 133.2, 132.8, 130.7, 128.8, 128.1, 127.7, 126.4, 124.2, 123.8, 123.4, 120.2, 111.4, 107.1, 67.7, 64.9, 64.0, 27.2, 26.6, 20.6; FTIR (neat): 2919, 2869, 1696, 1200 cm⁻¹; HRMS (ESI-TOF) m/z; calculated for C₂₃H₂₃O₄, [M+H]⁺ 363.1596, found 363.1591.

4-bromo-7,8,9,10-tetrahydro-6H,18H,20H-benzo[b]naphtho[2,3-g][1,5,9]trioxacyclotetradecin-18-one (4d): (40 mg, 60%) as a white solid, weight hourly space velocity (WHSV)=75 h⁻¹, space-time yield=20.51 g L⁻¹ h⁻¹, Melting point: 95-110° C.; ¹H NMR (400 MHz, CDCl₃) δ 8.28 (s, 1H), 7.81 (d, J=8.1 Hz, 1H), 7.66 (s, 1H), 7.60 (d, J=2.5 Hz, 1H), 7.47 (ddd, J=8.2, 6.9, 1.2 Hz, 1H), 7.41 (dd, J=8.7, 2.5 Hz, 1H), 7.34 (ddd, J=8.1, 6.9, 1.1 Hz, 1H), 7.08 (s, 1H), 6.78 (d, J=8.8 Hz, 1H), 5.36 (s, 2H), 4.15-4.04 (m, 4H), 2.00-1.87 (m, 4H), 1.79 (td, J=10.4, 5.5 Hz, 2H); ¹³C{¹H} NMR (100 MHz, CDCl₃) δ 168.3, 157.7, 154.9, 136.2, 135.8, 133.2, 133.0, 128.9, 128.3, 127.7, 126.4, 125.9, 124.3, 122.9, 113.2, 112.1, 107.2, 67.6, 65.4, 63.0, 27.1, 26.5, 20.5; FTIR (neat): 2921, 2839, 1725, 1240 cm⁻¹; HRMS (ESI-TOF) m/z; calculated for C₂₃H₂₁O₄BrNa, [M+Na]⁺ 463.0521, found 463.0514.

14-bromo-7,8,9,10-tetrahydro-6H,16H,18H-dibenzo[b,g][1,5,9]trioxacyclotetradecin-16-one (4e): (47.5 mg, 81%) as a sticky. weight hourly space velocity (WHSV)=66 h⁻¹, space-time yield=32.88 g L⁻¹ h⁻¹, ¹H NMR (400 MHz, CDCl₃) δ 7.87 (d, J=2.6 Hz, 1H), 7.44 (ddd, J=9.1, 8.1, 2.2 Hz, 2H), 7.32 (td, J=8.0, 1.7 Hz, 1H), 6.94-6.86 (m, 2H), 6.73 (d, J=8.8 Hz, 1H), 5.38 (s, 2H), 4.12 (t, J=5.1 Hz, 2H), 3.98-3.93 (m, 2H), 1.95-1.86 (m, 4H), 1.80-1.69 (m, 2H); ¹³C{¹H} NMR (100 MHz, CDCl₃) δ 167.0, 158.5, 157.3, 135.7, 134.4, 133.1, 130.7, 123.6, 123.1, 120.3, 114.5, 112.1, 111.4, 68.2, 64.8, 63.9, 27.1, 26.5, 20.4; FTIR (neat): 2921, 2870, 1674, 1600, 1299, 1261 cm⁻¹; HRMS (ESI-TOF) m/z; calculated for C₁₉H₁₉O₄BrNa, [M+Na]⁺ 413.0364, found 413.0358.

4-bromo-2-chloro-6,7,8,9-tetrahydro-15H,17H-dibenzo[b,g][1,5,9]trioxacyclotridecin-15-one (40): (32.4 mg, 51%) as a white solid weight hourly space velocity (WHSV)=72 h⁻¹, space-time yield=14.12 g L⁻¹ h⁻¹, ¹H NMR (400 MHz, CDCl₃) δ 7.74 (dd, J=7.7, 1.8 Hz, 1H), 7.56 (d, J=2.6 Hz, 1H), 7.45-7.39 (m, 2H), 7.00 (td, J=7.6, 0.9 Hz, 1H), 6.94 (d, J=8.3 Hz, 1H), 5.30 (s, 2H), 4.30 (t, J=7.2 Hz, 2H), 4.08 (t, J=5.4 Hz, 2H), 2.20-2.11 (m, 2H), 1.96 (dt, J=11.5, 5.5 Hz, 2H); ¹³C{¹H} NMR (100 MHz, CDCl₃) δ 168.0, 157.8, 155.9, 134.0, 133.5, 132.1, 131.9, 131.6, 129.4, 121.6, 121.0, 118.9, 113.8, 74.4, 69.2, 63.4, 27.8, 23.7; FTIR (neat): 2941, 2835, 11722, 1604, 1205 cm⁻¹; HRMS (ESI-TOF) m/z; calculated for C₁₈H₁₆BrClO₄Na, [M+Na]⁺ 432.9818, found 432.9810.

4-methyl-7,8,9,10-tetrahydro-6H,16H,18H-dibenzo[b,g][1,5,9]trioxacyclotetradecin-16-one (4g): (32.1 mg, 65%) as a semisolid, weight hourly space velocity (WHSV)=55 h⁻¹, space-time yield=17.84 g L⁻¹ h⁻¹, ¹H NMR (400 MHz, CDCl₃) δ 7.77 (dd, J=7.7, 1.5 Hz, 1H), 7.42-7.35 (m, 1H), 7.29-7.24 (m, 1H), 7.10 (dd, J=8.3, 1.7 Hz, 1H), 6.94 (t, J=7.5 Hz, 1H), 6.82 (dd, J=18.6, 8.3 Hz, 2H), 5.37 (s, 2H), 4.11 (t, J=5.2 Hz, 2H), 4.01-3.93 (m, 2H), 2.27 (s, 3H), 1.97-1.84 (m, 4H), 1.74 (td, J=10.5, 5.5 Hz, 2H); ¹³C{¹H} NMR (100 MHz, CDCl₃) δ 168.5, 158.2, 156.4, 133.8, 133.1, 131.9, 130.8, 129.4, 123.6, 121.3, 120.0, 112.6, 111.3, 67.8, 65.0, 63.5, 27.2, 26.7, 20.5, 20.4; FTIR (neat):2921, 2869, 1695, 1601, 1299, 1260 cm⁻¹; HRMS (ESI-TOF) m/z; calculated for C₂₀H₂₃O₄, [M+H]⁺ 327.1596, found 327.1588.

2-methoxy-6,7,8,9-tetrahydro-15H,17H-dibenzo[b,g][1,5,9]trioxacyclotridecin-15-one (4h): (43.5 mg, 85%) as a sticky, weight hourly space velocity (WHSV)=55 h⁻¹, space-time yield=31.60 g L⁻¹ h⁻¹, ¹H NMR (400 MHz, CDCl₃) δ 7.68 (dd, J=7.7, 1.7 Hz, 1H), 7.38 (ddd, J=8.2, 7.4, 1.8 Hz, 1H), 7.29 (d, J=8.3 Hz, 1H), 6.99 (td, J=7.6, 1.0 Hz, 1H), 6.95 (d, J=8.3 Hz, 1H), 6.52 (d, J=2.4 Hz, 1H), 6.45 (dd, J=8.3, 2.4 Hz, 1H), 5.30 (s, 2H), 4.16 (t, J=4.9 Hz, 2H), 4.06 (t, J=5.1 Hz, 2H), 3.79 (s, 3H), 2.08-1.93 (m, 4H); ¹³C{¹H} NMR (100 MHz, CDCl₃) δ 168.0, 161.7, 159.8, 158.5, 132.9, 131.1, 123.8, 121.5, 118.0, 116.9, 104.7, 101.5, 71.1, 69.0, 64.1, 55.5, 27.3, 26.5. FTIR (neat): 2924, 2831, 1735, 1606, 1236 cm⁻¹; HRMS (ESI-TOF) m/z; calculated for C₁₉H₂₀O₅Na, [M+Na]⁺ 351.1208, found 351.1200.

2,6,9-trioxa-1,7(1,2),4(1,3)-tribenzenacyclodecaphan-8-one (4i): (40.8 mg, 78%) as a semisolid, weight hourly space velocity (WHSV)=59 h⁻¹, space-time yield=27.22 g L⁻¹ h⁻¹, ¹H NMR (400 MHz, CDCl₃) δ 8.27 (s, 1H), 7.88 (dd, J=7.7, 1.8 Hz, 1H), 7.51-7.41 (m, 2H), 7.29-7.20 (m, 2H), 7.15 (d, J=7.5 Hz, 1H), 7.08-7.00 (m, 2H), 6.97 (d, J=8.3 Hz, 2H), 6.91 (td, J=7.4, 0.8 Hz, 1H), 5.62 (s, 2H), 5.41 (s, 2H), 5.11 (s, 2H); ¹³C{¹H} NMR (100 MHz, CDCl₃) δ 168.1, 157.5, 156.9, 138.0, 137.7, 133.5, 133.4, 132.3, 130.5, 127.8, 125.2, 125.1, 124.8, 124.1, 121.7, 121.3, 120.8, 113.3, 113.0, 69.3, 68.4, 62.3; FTIR (neat):2925, 2875, 1645, 1608, 1269, 1209 cm⁻¹; HRMS (ESI-TOF) m/z; calculated for C₂₂H₁₉O₄, [M+H]⁺ 347.1283, found 347.1288.

7,8,9,10-tetrahydro-6H,16H,18H-dibenzo[b,g][1,5]dioxa[9]thiacyclotetradecin-16-one (4j): (32.4 mg, 51%) as a white solid, weight hourly space velocity (WHSV)=55 h⁻¹, space-time yield=12.5 g L⁻¹ h⁻¹, Melting point: 106-105° C.; ¹H NMR (400 MHz, CDCl₃) δ 7.57 (dd, J=7.9, 0.8 Hz, 1H), 7.46 (dd, J=7.7, 1.4 Hz, 1H), 7.38-7.33 (m, 2H), 7.33-7.29 (m, 1H), 7.21 (td, J=7.6, 1.2 Hz, 1H), 6.94 (td, J=7.4, 0.9 Hz, 1H), 6.86 (d, J=8.2 Hz, 1H), 5.35 (s, 2H), 4.02-3.97 (m, 2H), 2.96 (t, J=5.8 Hz, 2H), 1.71 (dd, J=10.5, 5.7 Hz, 2H), 1.61 (dt, J=6.4, 3.2 Hz, 4H); ¹³C{¹H} NMR (100 MHz, CDCl₃) δ 168.3, 158.3, 137.8, 135.5, 135.0, 132.1, 130.8, 130.7, 128.4, 127.1, 123.8, 120.3, 111.6, 67.4, 64.9, 37.9, 28.9, 28.7, 24.4; FTIR (neat): 2923, 2837, 1722, 1607, 1202 cm⁻¹; HRMS (ESI-TOF) m/z; calculated for C₁₉H₂₁O_{3S}, [M+H]⁺ 329.1211, found 329.1207.

18-phenyl-7,8,9,10-tetrahydro-6H,16H,18H-dibenzo[b,g][1,5,9]trioxacyclotetradecin-16-one (4k): (32.41 mg, 51%) as a white solid, weight hourly space velocity (WHSV)=66 h⁻¹, space-time yield=29.22 g L⁻¹ h⁻¹, ¹H NMR (400 MHz, CDCl₃) δ 7.70 (dd, J=7.7, 1.7 Hz, 1H), 7.45 (d, J=7.4 Hz, 2H), 7.42-7.33 (m, 4H), 7.29 (d, J=7.1 Hz, 1H), 7.25 (dd, J=5.6, 0.8 Hz, 1H), 7.09 (dd, J=7.8, 1.6 Hz, 1H), 6.95 (dd, J=13.2, 7.9 Hz, 2H), 6.83 (dd, J=7.5, 6.1 Hz, 2H), 4.25-4.19 (m, 1H), 4.08-3.89 (m, 3H), 1.92-1.63 (m, 6H); ¹³C{¹H} NMR (100 MHz, CDCl₃) δ 166.8, 158.4, 157.9, 140.0, 132.8, 131.8, 130.9, 130.2, 128.2, 127.4, 127.2, 126.5, 122.7, 120.4, 119.8, 114.3, 111.2, 74.9, 69.0, 65.7, 27.4, 27.2, 21.3; FTIR (neat): 2925, 2829, 1720, 1601, 1210 cm⁻¹; HRMS (ESI-TOF) m/z; calculated for C₂₅H₂₄O₄Na, [M+Na]⁺ 411.1572 found 411.1572.

2-((5-(4-bromo-2-(1-hydroxyvinyl)phenoxy)pentyl)oxy)benzoic acid (41): (56 mg, 89%) as a semisolid, weight hourly space velocity (WHSV)=68 h⁻¹, space-time yield=42.5 g L⁻¹ h⁻¹, ¹H NMR (400 MHz, CDCl₃) δ 8.18 (dd, J=7.8, 1.8 Hz, 1H), 7.58-7.50 (m, 2H), 7.29 (dd, J=8.7, 2.5 Hz, 1H), 7.17-7.09 (m, 1H), 7.03 (d, J=8.3 Hz, 1H), 6.94 (dd, J=17.7, 11.2 Hz, 1H), 6.71 (d, J=8.8 Hz, 1H), 5.72 (dd, J=17.7, 1.2 Hz, 1H), 5.29 (dd, J=11.1, 1.1 Hz, 1H), 4.28 (t, J=6.5 Hz, 2H), 3.99 (t, J=6.1 Hz, 2H), 2.05-1.97 (m, 2H), 1.94-1.86 (m, 2H), 1.75-1.64 (m, 2H); OH peak have exchanged with deuterium of CDCl₃. ¹³C{¹H} NMR (100 MHz, CDCl₃) δ 165.5, 157.6, 155.1, 135.2, 133.9, 131.4, 130.5, 129.2, 129.0, 122.4, 117.8, 115.8, 113.66, 113.1, 112.7, 70.1, 68.1, 28.9, 28.8, 22.8; FTIR (neat):3356, 2928, 2841, 1725, 1601, 1212 cm⁻¹; FIRMS (ESI-TOF) m/z; calculated for C₂₀H₂₁O₄Br, [M+Na]⁺ 427.0521, found 427.0518.

18-methyl-7,8,9,10,17,18-hexahydro-6H,16H-dibenzo[b,g][1,9]dioxa[5]azacyclotetradecin-16-one (6a): Prepared using 2-((5-(2-(1-hydroxyethyl)phenoxy)pentyl)oxy)benzamide (28.2 mg, 0.08 mmol, 1 equiv.) dissolved in acetonitrile (2 mL, 0.05 M) as a solvent and flowed through the Omnifit® column reactor bed (6.6 mm bore×150 mm length) loaded with loaded with heterogeneous 2.43% Ru-Zeolite (1.5 gm, 8 cm bed height) with 0.1 mL/min flow rate to afford desired product 18-methyl-7,8,9,10,17,18-hexahydro-6H,16H-dibenzo[b,g][1,9]dioxa[5]azacyclotetradecin-16-one 6a (20 mg, 76% yield) as a white solid compound (20% ethyl acetate in n-hexane). Space time yield (STY)=10.57 gL⁻¹h⁻¹. ¹H NMR (400 MHz, CDCl₃) δ 8.89 (d, J=8.9 Hz, 1H), 8.30 (dd, J=7.9, 1.8 Hz, 1H), 7.40-7.35 (m, 1H), 7.32 (dd, J=7.3, 1.4 Hz, 1H), 7.23 (td, J=8.1, 1.7 Hz, 1H), 7.05-7.00 (m, 1H), 6.94-6.84 (m, 3H), 5.56 (dq, J=14.0, 7.0 Hz, 1H), 4.23-4.13 (m, 3H), 4.04-3.95 (m, 1H), 2.32-2.09 (m, 2H), 2.07-1.95 (m, 1H), 1.92-1.72 (m, 3H), 1.66 (d, J=7.0 Hz, 3H). ¹³C NMR (100 MHz, CDCl₃) δ 164.1, 157.2, 157.1, 132.8, 132.6, 130.8, 130.2, 128.7, 121.3, 120.9, 120.7, 111.4, 111.0, 67.7, 63.7, 50.1, 26.0, 25.3, 20.7, 20.7. IR (neat): 3355, 2990, 2929, 1657, 1601, 1550, 1240 cm⁻¹. FIRMS (ESI-TOF) m/z: [M+Na]⁺ calcd for C₂₀H₂₃NNaO₃, 348.1576 found: 348.1579.

16-methyl-7,8,15,16-tetrahydro-6H,14H-dibenzo[f,k][1,5]dioxa[9]azacyclododecin-14-one (6b): (19 mg, 80% yield) as a white solid compound (20% ethyl acetate in n-hexane). Space time yield (STY)=16.17 gL⁻¹h⁻¹. ¹H NMR (400 MHz, CDCl₃) δ 10.58 (d, J=8.9 Hz, 1H), 8.23 (dd, J=7.8, 1.8 Hz, 1H), 7.38 (td, J=8.2, 1.8 Hz, 1H), 7.21 (ddd, J=9.6, 5.4, 1.5 Hz, 2H), 7.06 (dd, J=11.1, 3.9 Hz, 1H), 6.91 (td, J=8.2, 2.1 Hz, 3H), 5.40-5.27 (m, 1H), 4.58 (ddd, J=12.6, 9.1, 3.9 Hz, 2H), 4.08-3.96 (m, 2H), 2.74-2.58 (m, 1H), 2.28-2.18 (m, 1H), 1.56 (d, J=7.0 Hz, 3H). ¹³C NMR (100 MHz, CDCl₃) δ 163.3, 156.9, 156.2, 132.3, 132.0, 131.3, 129.6, 128.2, 122.3, 121.4, 121.2, 111.9, 111.3, 68.3, 67.8, 49.6, 28.9, 22.8. IR (neat): 3365, 2997, 2935, 1662, 1598, 1535, 1220 cm⁻¹. HRMS (ESI-TOF) m/z: [M+Na]⁺ calcd for C₁₈H₁₉NNaO₃, 320.1262 found: 320.1263.

16-methyl-7,8,15,16-tetrahydro-6H,14H-dibenzo[f,k][1]oxa[5]thia[9]azacyclododecin-14-one (6c): (9 mg, 36% yield) as a white solid compound (20% ethyl acetate in n-hexane). Space time yield (STY)=3.44 gL⁻¹ h⁻¹. ¹H NMR (400 MHz, CDCl₃) δ 8.98 (d, J=6.4 Hz, 1H), 8.08 (dd, J=7.8, 1.5 Hz, 1H), 7.56 (dd, J=7.6, 1.2 Hz, 1H), 7.40 (td, J=7.6, 1.4 Hz, 1H), 7.37-7.29 (m, 2H), 7.28-7.23 (m, 1H), 7.00-6.88 (m, 2H), 5.38-5.27 (m, 1H), 4.39-4.27 (m, 2H), 3.17 (ddd, J=10.8, 6.1, 4.6 Hz, 1H), 3.07-2.96 (m, 1H), 2.31 (ddd, J=9.9, 7.8, 3.9 Hz, 1H), 2.10-1.99 (m, 1H), 1.68 (d, J=6.9 Hz, 3H). ¹³C NMR (100 MHz, CDCl₃) δ 165.7, 156.3, 137.6, 136.5, 131.9, 131.2, 130.7, 130.6, 129.4, 129.1, 128.8, 121.2, 112.5, 64.6, 49.3, 34.0, 25.5, 19.8. IR (neat): 3348, 2992, 2928, 1660, 1600, 1555, 1250 cm⁻¹. HRMS (ESI-TOF) m/z: [M+Na]⁺ calcd for C₁₈H₁₉NNaO₂S 336.1034 Found: 336.1031.

12-(2,4-difluorophenyl)-7,8,15,16-tetrahydro-6H,14H-dibenzo[f,k][1,5]dioxa[9]azacyclododecin-14-one (6d): (22 mg, 54% yield) as a white solid compound (20% ethyl acetate in n-hexane). Space time yield (STY)=12.63 gL⁻¹h⁻¹. ¹H NMR (400 MHz, CDCl₃) δ 10.24 (d, J=5.1 Hz, 1H), 8.35-8.28 (m, 1H), 7.55 (dt, J=8.5, 2.1 Hz, 1H), 7.40 (td, J=8.7, 6.5 Hz, 1H), 7.25 (td, J=9.3, 4.5 Hz, 2H), 7.01-6.84 (m, 5H), 4.68 (d, J=5.8 Hz, 2H), 4.32 (dd, J=9.1, 4.3 Hz, 4H), 2.46 (m, 2H). ¹³C NMR (100 MHz, CDCl₃) δ 164.0, 157.0, 156.4, 132.8 (d, J=4.1 Hz), 132.2, 131.5, 131.4 (dd, J=9.4, 4.9 Hz), 130.2, 128.6, 128.2, 126.4, 122.7, 121.1, 111.8, 111.7, 111.7, 111.5, 111.5, 104.4 (m), 68.8, 68.0, 41.8, 28.8. IR (neat): 3361, 2994, 2927, 1655, 1603, 1546, 1238, 966 cm⁻¹. HRMS (ESI-TOF) m/z: [M+H]⁺ calcd for C₂₃H₂₀F₂NO₃, 396.1411 found: 396.1407.

17-methyl-6,7,8,9,16,17-hexahydro-15H-dibenzo[b,g][1,9]dioxa[5]azacyclotridecin-15-one (6e): (12 mg, 46% yield) as a white solid compound (20% ethyl acetate in n-hexane). Space time yield (STY)=5.87 gL⁻¹h⁻¹. ¹H NMR (400 MHz, CDCl₃) δ 9.89 (d, J=9.1 Hz, 1H), 8.30 (dd, J=7.8, 1.8 Hz, 1H), 7.40 (ddd, J=8.3, 7.4, 1.9 Hz, 1H), 7.28-7.25 (m, 1H), 7.24-7.19 (m, 1H), 7.11-7.02 (m, 1H), 6.97 (d, J=8.0 Hz, 1H), 6.92 (ddd, J=7.4, 3.7, 1.0 Hz, 2H), 5.44 (dq, J=9.2, 6.9 Hz, 1H), 4.24-4.13 (m, 4H), 2.42-2.19 (m, 2H), 2.10-1.94 (m, 2H), 1.53 (d, J=6.9 Hz, 3H). ¹³C NMR (100 MHz, CDCl₃) δ 163.8, 157.6, 156.9, 132.6, 132.5, 131.6, 129.9, 128.3, 122.0, 121.4, 121.4, 113.0, 112.7, 69.9, 67.8, 50.0, 27.2, 26.7, 23.0. IR (neat): 3351, 2995, 2922, 1663, 1604, 1555, 1220 cm⁻¹. HRMS (ESI-TOF) m/z: [M+Na]+ calcd for C₁₉H₂₁NNaO₃, 334.1418 found: 334.1421.

18-phenyl-7,8,9,10,17,18-hexahydro-6H,16H-dibenzo[b,g][1,9]dioxa[5]azacyclotetradecin-16-one (6f): (23 mg, 74% yield) as a white solid compound (20% ethyl acetate in n-hexane). Space time yield (STY)=18.1 gL⁻¹h⁻¹. ¹H NMR (400 MHz, CDCl₃) δ 9.31 (d, J=9.3 Hz, 1H), 8.37 (dd, J=7.9, 1.8 Hz, 1H), 7.54 (dd, J=7.4, 1.6 Hz, 1H), 7.46-7.38 (m, 1H), 7.30 (td, J=8.0, 1.7 Hz, 1H), 7.27-7.19 (m, 4H), 7.15 (t, J=6.8 Hz, 1H), 7.07 (t, J=7.2 Hz, 1H), 6.99 (td, J=7.4, 0.8 Hz, 1H), 6.93 (d, J=8.3 Hz, 1H), 6.85 (d, J=8.1 Hz, 1H), 6.73 (d, J=9.4 Hz, 1H), 4.28-4.18 (m, 1H), 4.03 (dd, J=11.8, 7.3 Hz, 2H), 3.78 (dt, J=8.9, 4.4 Hz, 1H), 2.11-1.88 (m, 2H), 1.80-1.67 (m, 2H), 1.66-1.58 (m, 2H). ¹³C NMR (100 MHz, CDCl₃) δ 164.8, 157.4, 156.8, 142.2, 133.1, 132.9, 131.8, 129.3, 129.2, 127.8, 126.1, 125.8, 121.0, 120.9, 120.6, 111.5, 111.3, 67.8, 63.6, 56.1, 25.5, 25.1, 20.4. IR (neat): 3395, 2994, 1641, 1493, 1252, 1180 cm⁻¹. HRMS (ESI-TOF) m/z: [M+Na]⁺ calcd for C₂₅H₂₅NNaO₃, 388.1912 found: 388.1916.

3-methoxy-20-phenyl-7,8,9,10,19,20-hexahydro-6H,18H-benzo[b]naphtho[2,3-g][1,9]dioxa[5]azacyclotetradecin-18-one (6g): (14 mg, 35% yield) as a white solid compound (20% ethyl acetate in n-hexane). Space time yield (STY)=5.21 gL⁻¹h⁻¹. ¹H NMR (400 MHz, CDCl₃) δ 9.36 (d, J=9.3 Hz, 1H), 8.92 (s, 1H), 7.90 (d, J=8.1 Hz, 1H), 7.71 (d, J=8.2 Hz, 1H), 7.54-7.44 (m, 2H), 7.40-7.33 (m, 1H), 7.32-7.08 (m, 7H), 6.72 (d, J=9.3 Hz, 1H), 6.50 (dd, J=8.2, 2.4 Hz, 1H), 6.45 (d, J=2.3 Hz, 1H), 4.38-4.27 (m, 1H), 4.12 (dd, J=11.7, 5.9 Hz, 1H), 4.01 (td, J=9.4, 5.0 Hz, 1H), 3.86-3.74 (m, 4H), 2.13-1.91 (m, 2H), 1.62 (m, 4H). ¹³C NMR (100 MHz, CDCl₃) δ 164.59, 160.92, 157.85, 154.77, 142.51, 135.93, 134.88, 132.25, 129.43, 128.33, 128.30, 127.84, 126.17, 126.11, 125.85, 124.44, 122.10, 122.04, 106.71, 103.61, 99.83, 77.16, 68.06, 63.78, 55.72, 55.55, 25.52, 25.16, 20.60. IR (neat): 3386, 2994, 1648, 1504, 1252, 1177 cm⁻¹. HRMS (ESI-TOF) m/z: [M+H]⁺ calcd for C₃₀H₃₀NO₄, 468.2175 found: 468.2177.

2,3,20-trimethyl-7,8,9,10,19,20-hexahydro-6H,18H-benzo[b]naphtho[2,3-g][1,9]dioxa[5]azacyclotetradecin-18-one (6h): (11 mg, 57% yield) as a brownish sticky compound (20% ethyl acetate in n-hexane). Space time yield (STY)=6.67 gL⁻¹h⁻¹. ¹H NMR (400 MHz, CDCl₃) δ 9.55 (d, J=8.7 Hz, 1H), 8.82 (s, 1H), 7.88 (d, J=8.1 Hz, 1H), 7.70 (d, J=8.2 Hz, 1H), 7.52-7.43 (m, 1H), 7.36 (dd, J=11.1, 3.9 Hz, 1H), 7.19 (s, 1H), 6.94 (d, J=15.6 Hz, 2H), 5.50 (dq, J=14.1, 7.0 Hz, 1H), 4.38-4.30 (m, 1H), 4.25-4.10 (m, 3H), 2.31 (s, 3H), 2.26 (s, 3H), 2.02 (dd, J=14.1, 8.7 Hz, 6H), 1.64 (d, J=7.0 Hz, 3H). ¹³C NMR (100 MHz, CDCl₃) δ 164.2, 154.9, 153.7, 135.7, 135.0, 134.1, 133.4, 131.6, 131.2, 129.2, 128.7, 128.2, 128.0, 126.2, 124.3, 123.0, 106.7, 73.7, 69.5, 50.4, 28.1, 27.8, 24.9, 24.5, 20.6, 16.9. IR (neat): 3371, 2985, 2921, 1661, 1605, 1545, 1232 cm⁻¹. HRMS (ESI-TOF) m/z: [M+H]⁺ calcd for C₂₆H₃₀NO₃, 404.2225 found: 404.2221.

4-fluoro-20-methyl-7,8,9,10,19,20-hexahydro-6H,18H-benzo[b]naphtho[2,3-g][1,9]dioxa[5]azacyclotetradecin-18-one (6i): (15 mg, 44% yield) as a white solid compound (20% ethyl acetate in n-hexane). Space time yield (STY)=7.02 gL⁻¹h⁻¹. ¹H NMR (400 MHz, CDCl₃) δ 8.98 (d, J=9.2 Hz, 1H), 8.85 (s, 1H), 7.88 (d, J=8.1 Hz, 1H), 7.68 (d, J=8.2 Hz, 1H), 7.47 (t, J=7.5 Hz, 1H), 7.35 (t, J=7.5 Hz, 1H), 7.16-7.07 (m, 2H), 6.95-6.87 (m, 1H), 6.81 (dd, J=8.9, 4.4 Hz, 1H), 5.55 (dq, J=9.1, 7.0 Hz, 1H), 4.34-4.26 (m, 1H), 4.13 (dd, J=8.1, 3.1 Hz, 3H), 2.30-2.12 (m, 2H), 1.94-1.72 (m, 4H), 1.68 (d, J=7.0 Hz, 3H). ¹³C NMR (100 MHz, CDCl₃) δ 164.1, 157.9, 155.5, 154.5, 153.4, 153.3, 135.8, 134.5, 131.6 (d, J=6.6 Hz), 129.4, 128.4, 126.1, 124.4, 122.3, 117.7 (d, J=23.2 Hz), 114.2 (d, J=22.5 Hz), 111.5 (d, J=8 Hz), 106.6, 67.9, 64.3, 49.8, 26.0, 25.3, 20.8, 20.5. IR (neat): 3345, 2990, 2930, 1655, 1597, 1210, 985 cm⁻¹. HRMS (ESI-TOF) m/z: [M+H]⁺ calcd for C₂₄H₂₅FNO₃, 394.1818 found: 394.1813.

2,4,20-trimethyl-7,8,9,10,19,20-hexahydro-6H,18H-benzo[b]naphtho[2,3-g][1,9]dioxa[5]azacyclotetradecin-18-one (6j): (14 mg, 49% yield) as a colourless oily compound (20% ethyl acetate in n-hexane). Space time yield (STY)=7.29 gL⁻¹h⁻¹. ¹H NMR (400 MHz, CDCl₃) δ 9.55 (d, J=8.7 Hz, 1H), 8.82 (s, 1H), 7.88 (d, J=8.1 Hz, 1H), 7.70 (d, J=8.1 Hz, 1H), 7.52-7.44 (m, 1H), 7.41-7.30 (m, 1H), 7.19 (s, 1H), 6.94 (d, J=15.1 Hz, 2H), 5.50 (dq, J=14.1, 7.0 Hz, 1H), 4.38-4.31 (m, 1H), 4.26-4.12 (m, 3H), 2.31 (s, 3H), 2.26 (s, 3H), 2.12-1.93 (m, 6H), 1.63 (d, J=7.0 Hz, 3H). ¹³C NMR (100 MHz, CDCl₃) δ 164.2, 154.9, 153.7, 135.8, 135.0, 134.1, 133.5, 131.6, 131.2, 129.3, 128.7, 128.2, 128.0, 126.2, 124.3, 123.0, 106.7, 73.7, 69.5, 50.4, 28.1, 27.9, 24.9, 24.5, 20.7, 17.0. IR (neat): 3368, 2982, 2923, 1662, 1604, 1548, 1235 cm⁻¹. HRMS (ESI-TOF) m/z: [M+H]⁺ calcd for C₂₂H₂₈NO₄, 370.2018 found: 370.2017.

7,8,9,10,19,20-hexahydro-6H,18H-benzo[b]naphtho[2,3-g][1,9]dioxa[5]azacyclotetradecin-18-one (6k): (5 mg, 17% yield) as a white solid compound (20% ethyl acetate in n-hexane). Space time yield (STY)=0.9 gL⁻¹h⁻¹. ¹H NMR (400 MHz, CDCl₃) δ 8.87 (s, 1H), 8.82 (s, 1H), 7.89 (d, J=8.3 Hz, 1H), 7.68 (d, J=8.1 Hz, 1H), 7.51-7.43 (m, 2H), 7.40-7.33 (m, 1H), 7.28 (dd, J=7.9, 1.7 Hz, 1H), 7.12 (s, 1H), 6.96-6.88 (m, 2H), 4.75 (d, J=6.0 Hz, 2H), 4.19 (dd, J=7.5, 4.2 Hz, 4H), 2.04-1.97 (m, 4H), 1.94 (m, 2H). ¹³C NMR (100 MHz, CDCl₃) δ 164.6, 157.4, 154.5, 135.8, 134.2, 131.4, 129.4, 129.2, 128.3, 128.2, 126.7, 126.1, 124.4, 122.4, 120.7, 110.4, 106.6, 68.1, 63.6, 40.2, 26.2, 25.6, 20.8. IR (neat): 3345, 2995, 1653, 1526, 1181 cm⁻¹. HRMS (ESI-TOF) m/z: [M+H]⁺ calcd for C₂₃H₂₄NO₃, 362.1756 found: 362.1751.

6,7,9,10,17,18-hexahydro-16H-dibenzo[h,m][1,4,7]trioxa[11]azacyclotetradecin-16-one (61): (8 mg, 32% yield) as a yellowish sticky compound (30% ethyl acetate in n-hexane). Space time yield (STY)=2.72 gL⁻¹h⁻¹. ¹H NMR (400 MHz, CDCl₃) δ 8.77 (s, 1H), 8.23 (dd, J=7.8, 1.8 Hz, 1H), 7.44 (dd, J=7.3, 1.6 Hz, 1H), 7.37 (td, J=7.1, 1.8 Hz, 1H), 7.23 (td, J=8.0, 1.7 Hz, 1H), 7.06-7.00 (m, 1H), 6.91 (td, J=7.5, 0.7 Hz, 1H), 6.85 (d, J=8.2 Hz, 1H), 6.81 (d, J=8.1 Hz, 1H), 4.71 (d, J=6.2 Hz, 2H), 4.19 (dd, J=4.2, 2.6 Hz, 4H), 4.02-3.98 (m, 2H), 3.97-3.92 (m, 2H). ¹³C NMR (100 MHz, CDCl₃) δ 164.8, 157.3, 156.8, 132.8, 132.5, 131.2, 128.9, 127.5, 121.8, 121.2, 120.9, 111.5, 110.6, 69.4, 68.7, 67.5, 66.0, 39.7. IR (neat): 3443, 2930, 1665, 1595, 1201, 1165 cm⁻¹. HRMS (ESI-TOF) m/z: [M+H]⁺ calcd for C₁₈H₁₉NNaO₄, 336.1211 found: 336.1213.

2-methoxy-19-methyl-6,7,8,9,10,11,18,19-octahydro-17H-dibenzo[b,g][1,9]dioxa[5]azacyclopentadecin-17-one (6m): (6 mg, 34% yield) as a white solid compound (20% ethyl acetate in n-hexane). Space time yield (STY)=2.17 gL⁻¹h⁻¹. ¹H NMR (400 MHz, CDCl₃) δ 8.61 (d, J=8.7 Hz, 1H), 8.20 (dd, J=7.8, 1.8 Hz, 1H), 7.37 (ddd, J=8.4, 7.4, 1.9 Hz, 1H), 7.03-6.98 (m, 1H), 6.95 (d, J=3.1 Hz, 1H), 6.91 (d, J=8.5 Hz, 1H), 6.82 (d, J=8.9 Hz, 1H), 6.72 (dd, J=8.9, 3.1 Hz, 1H), 5.38 (dq, J=14.3, 7.1 Hz, 1H), 4.21 (t, J=5.0 Hz, 2H), 4.06 (t, J=3.8 Hz, 2H), 3.76 (s, 3H), 2.01-1.89 (m, 4H), 1.88-1.77 (m, 4H), 1.70 (d, J=7.1 Hz, 3H). ¹³C NMR (100 MHz, CDCl₃) δ 164.3, 157.0, 153.5, 151.9, 132.7, 132.5, 132.1, 121.9, 120.9, 116.5, 113.7, 113.2, 111.7, 69.9, 68.7, 55.8, 49.9, 28.0, 28.0, 26.7, 26.0, 20.5. IR (neat): 3403, 2992, 1644, 1598, 1221, 1173 cm⁻¹. HRMS (ESI-TOF) m/z: [M+H]⁺ calcd for C₂₂H₂₈NO₄, 370.2018 found: 370.2017.

2-chloro-19-methyl-6,7,8,9,10,11,18,19-octahydro-17H-dibenzo[b,g][1,9]dioxa[5]azacyclopentadecin-17-one (6n): (7 mg, 23% yield) as a white solid compound (20% ethyl acetate in n-hexane). Space time yield (STY)=1.71 gL⁻¹h⁻¹. ¹H NMR (400 MHz, CDCl₃) δ 8.59 (d, J=7.8 Hz, 1H), 8.20 (dd, J=7.8, 1.7 Hz, 1H), 7.37 (d, J=2.8 Hz, 2H), 7.14 (dd, J=8.7, 2.6 Hz, 1H), 7.01 (t, J=7.4 Hz, 1H), 6.92 (d, J=8.3 Hz, 1H), 6.79 (d, J=8.7 Hz, 1H), 5.35 (dd, J=15.5, 7.4 Hz, 1H), 4.24 (t, J=5.0 Hz, 2H), 4.07 (ddd, J=17.5, 9.1, 3.2 Hz, 2H), 2.00-1.73 (m, 8H), 1.69 (d, J=7.2 Hz, 3H). ¹³C NMR (100 MHz, CDCl₃) δ 164.4, 156.9, 156.3, 132.8, 132.7, 132.6, 130.8, 128.1, 125.5, 121.7, 121.0, 113.5, 111.7, 69.3, 68.5, 49.4, 27.9, 26.4, 25.9, 20.1. IR (neat): 3400, 2940, 1650, 1585, 1204 cm⁻¹. HRMS (ESI-TOF) m/z: [M+H]⁺ calcd for C₂₁H₂₆ClNO₃ 374.1523 found: 374.1530.

13-(2,4-difluorophenyl)-17-methyl-6,7,8,9,16,17-hexahydro-15H-dibenzo[b,g][1,9]dioxa[5]azacyclotridecin-15-one (60): (14 mg, 52% yield) as a yellowish liquid compound (20% ethyl acetate in n-hexane). Space time yield (STY)=7.74 gL⁻¹h⁻¹. ¹H NMR (400 MHz, CDCl₃) δ 9.91 (d, J=9.2 Hz, 1H), 8.42 (d, J=2.0 Hz, 1H), 7.58 (dt, J=8.5, 2.3 Hz, 1H), 7.43 (td, J=8.7, 6.5 Hz, 1H), 7.28 (d, J=1.5 Hz, 1H), 7.22 (dd, J=11.6, 4.1 Hz, 1H), 7.02-6.85 (m, 4H), 5.45 (dd, J=9.2, 6.9 Hz, 1H), 4.31-4.15 (m, 4H), 2.48-2.20 (m, 2H), 2.14-1.97 (m, 2H), 1.54 (d, J=6.9 Hz, 3H). ¹³C NMR (100 MHz, CDCl₃) δ 163.5, 157.1, 156.9, 133.1, 132.8, 131.5, 130.0, 128.4, 128.2, 122.2, 121.5, 113.0, 112.8, 111.6 (m), 11.4 (m), 104.6, 104.4, 104.1 (m), 70.2, 67.8, 50.1, 27.1, 26.7, 23.0. IR (neat): 3405, 2945, 1656, 1580, 1215 cm⁻¹. HRMS (ESI-TOF) m/z: [M+H]⁺ calcd for C₂₁H₂₆ClNO₃ 374.1523 found: 374.1530. A skilled artisan will appreciate that the quantity and type of each ingredient can be used in different combinations or singly. All such variations and combinations would be falling within the scope of present disclosure.

The foregoing examples are merely illustrative and are not to be taken as limitations upon the scope of the invention. Various changes and modifications to the disclosed embodiments will be apparent to those skilled in the art. Such changes and modifications may be made without departing from the scope of the invention.

### ADVANTAGES OF THE PRESENT INVENTION

The present invention is to provide a continuous-flow catalytic dehydration process for the synthesis of macrocyclic compounds.

The present invention is to provide a process for the synthesis of macrocyclic compounds that include heterogeneous catalysis.

The present invention is to provide a process for the synthesis of macrocyclic compounds that are scalable to grams.

The present invention is to provide a process for the synthesis of macrocyclic compounds with diverse substrates with different ring sizes.

The present invention is to provide a process for the synthesis of macrocyclic compounds with water as a by-product.

The present invention is to provide a process for the synthesis of macrocyclic compounds with no requirement of predefined substrates.

The present invention is to provide a process for the synthesis of macrocyclic compounds with no special reaction conditions.

We claim:

1. A continuous flow process for preparation of macrocyclic compound of formula (I), 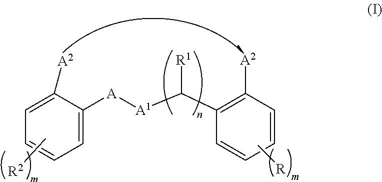
wherein:
A is CH₂ or CO;
A¹ is O or NH;
A² is O or S;
R is hydrogen, OH, C₁₋₆alkyl, halogen, substituted or unsubstituted phenyl, or C₁₋₆alkyloxy; or R along with hydrogen of phenyl ring form naphthyl ring, wherein the substitution is one or more group selected from halogen or OH;
R¹ is hydrogen, or C₁₋₆alkyl;
R² is hydrogen, OH, C₁₋₆alkyl, halogen, substituted or unsubstituted phenyl, or C₁₋₆alkyloxy, or R² along with hydrogen of phenyl ring form naphthyl ring, wherein the substitution is one or more group selected from halogen or OH;
n is 0, 1, 2, 3, 4 or 5;
m is 0, 1 or 2; and [symbol]
represents C₂₋₁₄ linear alkyl; wherein the one or more CH₂ of C₂₋₁₄ linear alkyl is optionally replaced with O or S.
wherein the process comprises the steps of:
dissolving a compound of formula (Ia) (wherein X is CH₂OH, COOH or CONH₂; A², R, R¹, R², n and m are as defined above) in a solvent to obtain a solution; and 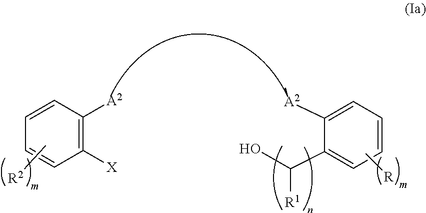
reacting the solution with a heterogeneous catalyst present in a continuous flow reactor at a temperature in a range of 100° C. to 150° C. with the flow rate in a range of 0.05 mL/min to 2 mL/min at back pressure in a range of 2.5 to 4 bar to obtain the compound of formula (I).

2. The continuous flow process as claimed in claim 1, wherein the solvent is selected from the group consisting of toluene, 1,4-dioxane, acetonitrile, tetrahydrofuran, 2-methyltetrahydrofuran, dimethyl sulfoxide, dimethylformamide, dimethyl carnonate, and diethylene glycol dimethyl ether.

3. The continuous flow process as claimed in claim 1, wherein the heterogeneous catalyst is β-Zeolite, Amberlyst-15, Amberlyst-35, Nafion, montmorillonite clay, Dowex 50. Ni-Zeolite, Fe-Zeolite, Ru-Zeolite, NiCl₂·6H₂O, RuCl₃·xH₂O, SnCl₂, ZnCl₂, FeCl₃, Fe(OTf)₃, TiCl₄, InCl₃, and Mn(OAc)₃

4. The continuous flow process as claimed in claim 1, wherein the temperature of the reaction is in a range of 115° C. to 125° C.

5. The continuous flow process as claimed in claim 1, wherein the flow rate is 0.1 mL/min.

6. The continuous flow process as claimed in claim 1, wherein the back pressure is in a range of 3.3 to 3.5 bar.

7. The continuous flow process as claimed in claim 1, wherein when X represents CH₂OH in the compound of formula (Ia) then A is CH₂ and A¹ is O in the compound of formula (I).

8. The continuous flow process as claimed in claim 1, wherein when X represents COOH in the compound of formula (Ia) then A¹ is CO and A¹ is O in the compound of formula (I).

9. The continuous flow process as claimed in claim 1, wherein when X represents CONH₂ in the compound of formula (Ia) then A is CO and A¹ is NH in the compound of formula (I).

10. The continuous flow process as claimed in claim 1, wherein the [symbol] represents C₂₋₁₄ linear alkyl; wherein one or more CH₂ of C₂₋₁₄ linear alkyl is optionally replaced with O.

11. The continuous flow process as claimed in claim 1, wherein the compound of formula (I) is selected from the group consisting of: 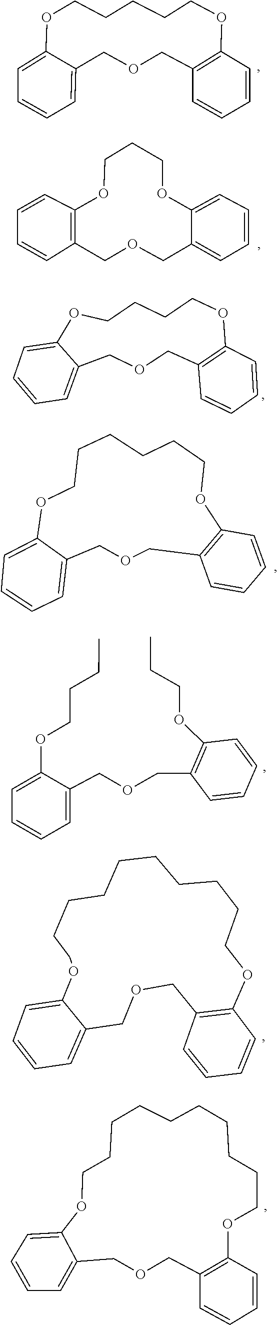 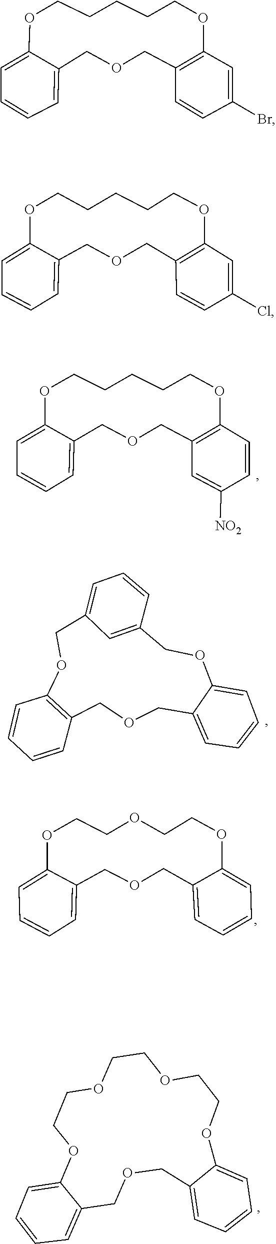 [SMILES: Brc1ccc2c(c1)OCCCCCOc1ccccc1COC2 ; Clc1ccc2c(c1)OCCCCCOc1ccccc1COC2 ; O=[N+]([O-])c1ccc2c(c1)COCc1ccccc1OCCCCCO2 ; c1cc2cc(c1)COc1ccccc1COCc1ccccc1OC2 ; c1ccc2c(c1)COCc1ccccc1OCCOCCO2 ; c1ccc2c(c1)COCc1ccccc1OCCOCCOCCO2] 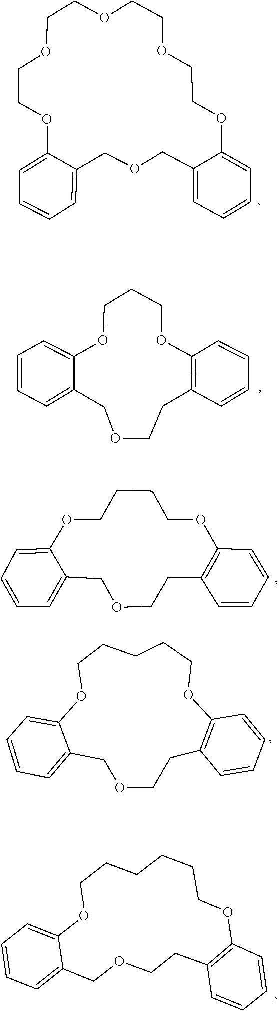 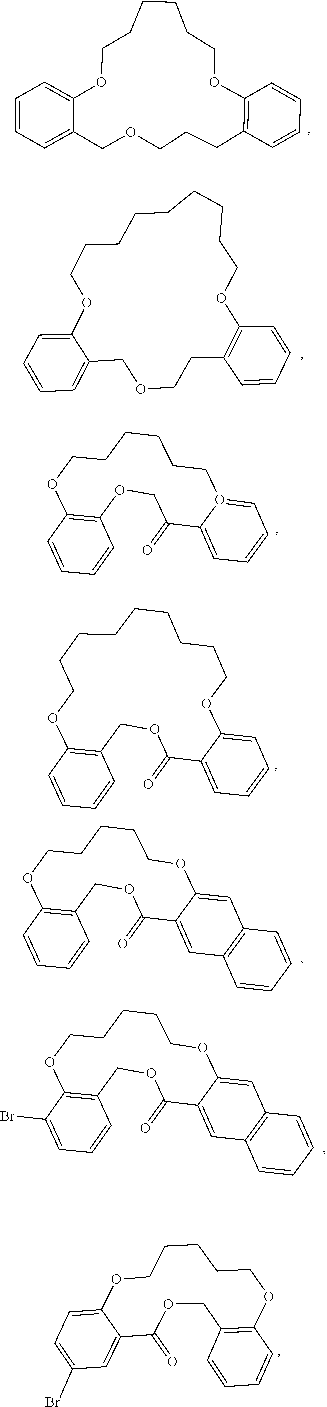 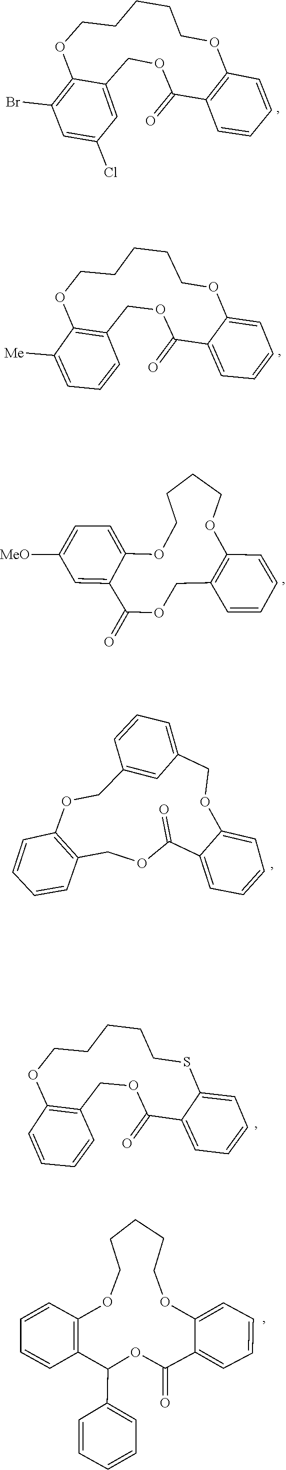 [SMILES: O=C1OCc2cc(Cl)cc(Br)c2OCCCCCOc2ccccc21 ; Cc1cccc2c1OCCCCCOc1ccccc1C(=O)OC2 ; COc1ccc2c(c1)C(=O)OCc1ccccc1OCCCCO2 ; O=C1OCc2ccccc2OCc2cccc(c2)COc2ccccc21 ; O=C1OCc2ccccc2OCCCCCSc2ccccc21 ; O=C1OC(c2ccccc2)c2ccccc2OCCCCCOc2ccccc21] 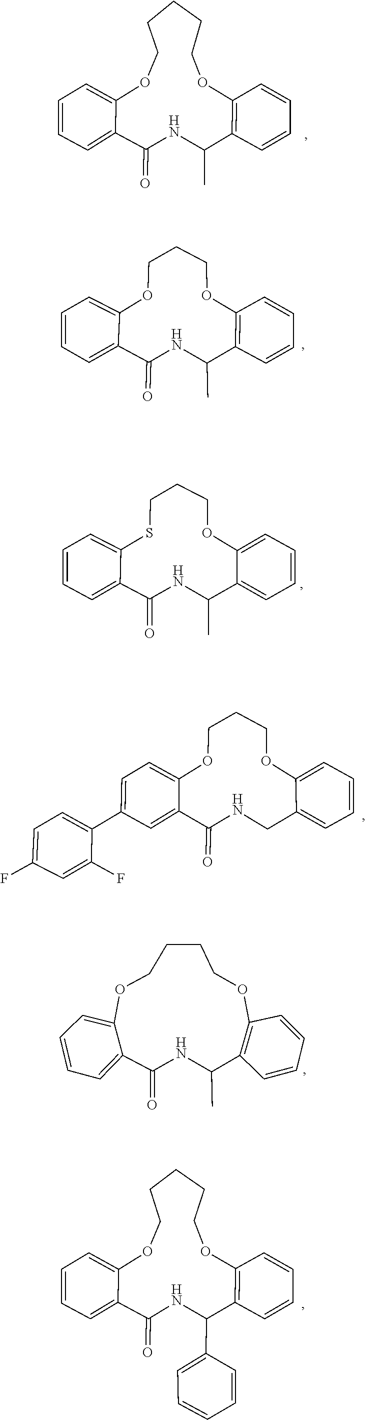 [SMILES: CC1NC(=O)c2ccccc2OCCCCCOc2ccccc21 ; CC1NC(=O)c2ccccc2OCCCOc2ccccc21 ; CC1NC(=O)c2ccccc2SCCCOc2ccccc21 ; O=C1NCc2ccccc2OCCCOc2ccc(-c3ccc(F)cc3F)cc21 ; CC1NC(=O)c2ccccc2OCCCCOc2ccccc21 ; O=C1NC(c2ccccc2)c2ccccc2OCCCCCOc2ccccc21] 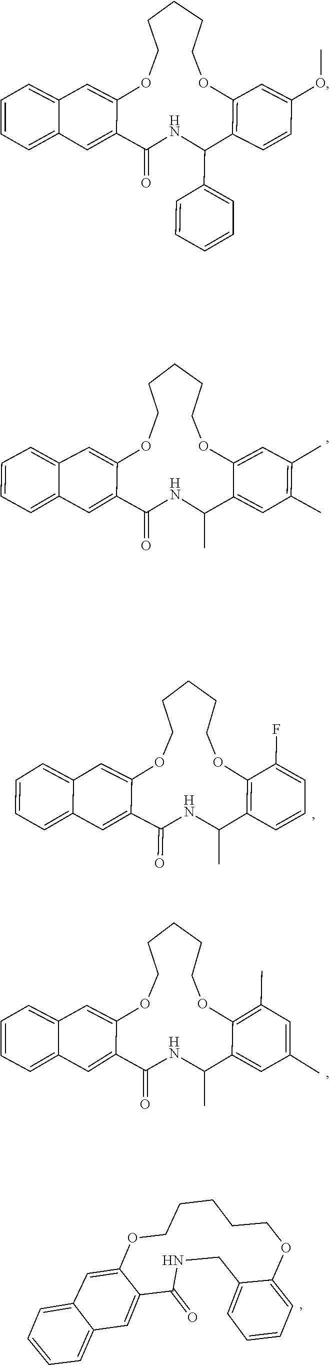 [SMILES: COc1ccc2c(c1)OCCCCCOc1cc3ccccc3cc1C(=O)NC2c1ccccc1 ; Cc1cc2c(cc1C)C(C)NC(=O)c1cc3ccccc3cc1OCCCCCO2 ; CC1NC(=O)c2cc3ccccc3cc2OCCCCCOc2c(F)cccc21 ; Cc1cc(C)c2c(c1)C(C)NC(=O)c1cc3ccccc3cc1OCCCCCO2 ; O=C1NCc2ccccc2OCCCCCOc2cc3ccccc3cc21] 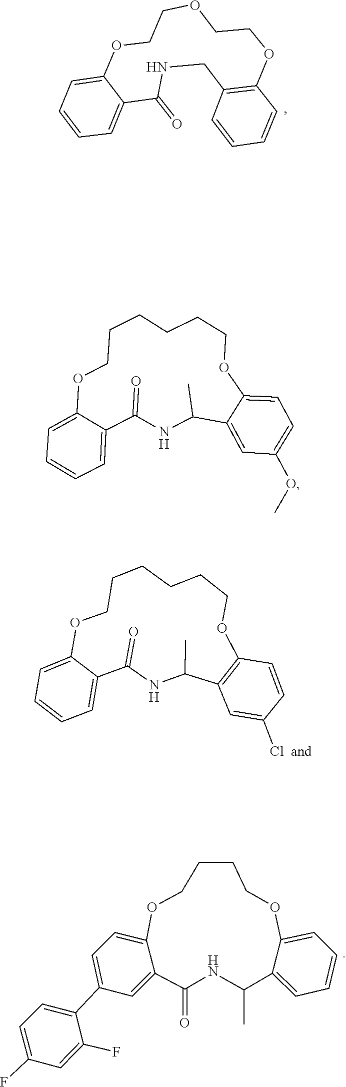 [SMILES: O=C1NCc2ccccc2OCCOCCOc2ccccc21 ; COc1ccc2c(c1)C(C)NC(=O)c1ccccc1OCCCCCCO2 ; CC1NC(=O)c2ccccc2OCCCCCCOc2ccc(Cl)cc21 ; CC1NC(=O)c2cc(-c3ccc(F)cc3F)ccc2OCCCCOc2ccccc21]
